(19) United States
(12) Patent Application Publication
(10) Pub. No.: US 2026/0315623 A1
(43) Pub. Date: Oct. 15, 2026

(54) Extracellular Vesicle-Associated Adeno-Associated Virus Vectors for Inhaled Gene Therapy

(71) Applicant: The Johns Hopkins University, Baltimore, MD (US)

(72) Inventors: Jung Soo Suk, Clarksville, MD (US); Gijung Kwak, Baltimore, MD (US)

(21) Appl. No.: 19/100,402

(22) PCT Filed: Aug. 2, 2023
(86) PCT No.: PCT/US2023/029349

§ 371 Date: Jan. 31, 2025

Related U.S. Application Data
(60) Provisional application No. 63/394,498, filed on Aug. 2, 2022.

(51) Int. Cl. A61K 48/00; A61K 38/08; A61P 11/06; C07K 14/005; C12N 15/86

(52) U.S. Cl. CPC A61K 48/0033; A61K 38/08; A61K 48/005; A61K 48/0075; A61P 11/06; C07K 14/005; C12N 15/86; C12N 2750/14122; C12N 2750/14143

(57) ABSTRACT

An extracellular vesicle (EV) associated with a viral vector is provided. The combination of the EV and virus vectors provide widespread and highly efficient transgene expression in lungs, following localized administration, as well as in mucus-covered air-liquid interface (ALI) cultures with primary human bronchial epithelial (HBE) cells and nasal epithelial (HNE) cells.

### CROSS-REFERENCE TO RELATED APPLICATION

The present application claims priority of U.S. provisional application No. 63/394,498 filed Aug. 2, 2022, which is incorporated by reference herein in its entirety.

### STATEMENT REGARDING FEDERALLY SPONSORED RESEARCH

This invention was made with government support under grant NS111102 awarded by the National Institutes of Health. The government has certain rights in the invention.

### BACKGROUND

Gene therapy using adeno-associated virus (AAV) is an emerging treatment modality, including for treatment of single-gene defects. An example of one such disease is cystic fibrosis. Cystic fibrosis (CF) is a lethal, autosomal-recessive disorder that affects at least 30,000 people in the U.S. alone, and at least 70,000 people worldwide. The average survival age for CF patients is about 40 years. CF is caused by mutations in the gene encoding the cystic fibrosis transmembrane conductance regulator (CFTR), a channel that conducts chloride and bicarbonate ions across epithelial cell membranes. Impaired CFTR function leads to inflammation of the airways and progressive bronchiectasis. Because of the single-gene etiology of CF and the various CFTR mutations in the patient population, gene therapy potentially provides a universal cure for CF.

Adeno-associated virus (AAV), a member of the human parvovirus family, is a non-pathogenic virus that depends on helper viruses for its replication. For this reason, recombinant AAV (rAAV) vectors are among the most frequently used in gene therapy pre-clinical studies and clinical trials.

### SUMMARY

Compositions for achieving therapeutically relevant gene transfer efficacy in the lung following localized administration are provided.

Accordingly, in certain aspects, a method of delivery of nucleic acid (e.g. a transgene), through a mucus gel layer comprises administering a composition comprising a virus vector associated with an extracellular vesicle, wherein the virus vector comprises the nucleic acid. In certain embodiments, the virus vector is associated externally with the extracellular vesicle or is contained within the extracellular vesicle. In certain embodiments, the virus vector comprises: adenovirus vectors, adeno-associated virus (AAV) vectors, recombinant adeno-associated virus (rAAV) vectors, herpes simplex virus vectors, influenza virus vectors, lentivirus vector, retrovirus vectors, alphavirus vectors, flavivirus vectors, rhabdovirus vectors, measles virus vectors, Newcastle disease virus vectors, poxvirus vectors, vaccinia virus vectors, modified Ankara virus vectors, vesicular stomatitis virus vectors, picornavirus vectors, chimeric virus vectors, synthetic virus vectors, respiratory syncytial virus vectors, parainfluenza virus vectors, foamy virus vectors, recombinant viral vectors, mosaic virus vectors, or pseudotyped virus vectors. In certain embodiments, the virus vector comprises one or more mutations. In certain embodiments, the virus vector is an AAV vector. In certain embodiments, the AAV vector comprises one or more AAV capsid proteins comprising modified amino acid sequences. In certain embodiments, the modified amino acid capsid proteins comprise one or more insertions, deletions, or substitutions of one or more amino acids. In certain embodiments, the one or more capsid amino acid mutations enhance intracellular trafficking to a target cell nucleus as compared to an AAV lacking the capsid amino acid mutations. In certain embodiments, the capsid amino acid mutations enhance the interactions with target cells and/or the penetration through the mucus gel layer as compared to an AAV lacking the capsid amino acid mutations. In certain embodiments, the AAV comprises AAV serotypes 1, 2, 3, 4, 5, 6, 7, 8, 9, 10, 11, 12, 13, 14, 15, 16, DJ, DJ/8, or mutants thereof. In certain embodiments, the AAV is AAV serotype 6 (AAV6). In certain embodiments, the extracellular vesicle is derived from eukaryotic cells, such as mammalian cells or insect cells. In certain embodiments, the composition is formulated for oral, intratracheal/oropharyngeal, cervicovaginal, ocular or nasal delivery. In certain embodiments, the composition is aerosolized for delivery to the airways by inhalation or the oropharyngeal cavity. In certain embodiments, the composition is formulated as a liquid or gel for delivery to mucosal surfaces. In certain embodiments, the composition is formulated as a suppository.

In another aspect, a composition comprises a virus vector associated with an extracellular vesicle, wherein the virus vector comprises the nucleic acid. In certain embodiments, the virus vector is associated externally with the extracellular vesicle or is contained within the extracellular vesicle. In certain embodiments, the virus vector comprises: adenovirus vectors, adeno-associated virus (AAV) vectors, recombinant adeno-associated virus (rAAV) vectors, herpes simplex virus vectors, influenza virus vectors, lentivirus vector, retrovirus vectors, alphavirus vectors, flavivirus vectors, rhabdovirus vectors, measles virus vectors, Newcastle disease virus vectors, poxvirus vectors, vaccinia virus vectors, modified Ankara virus vectors, vesicular stomatitis virus vectors, picornavirus vectors, chimeric virus vectors, synthetic virus vectors, respiratory syncytial virus vectors, parainfluenza virus vectors, foamy virus vectors, recombinant viral vectors, mosaic virus vectors, or pseudotyped virus vectors. In certain embodiments, the virus vector is an AAV vector. In certain embodiments, the AAV vector comprises one or more AAV capsid proteins comprising modified amino acid sequences. In certain embodiments, the modified amino acid capsid proteins comprise one or more insertions, deletions, or substitutions of one or more amino acids. In certain embodiments, the one or more capsid amino acid mutations enhance intracellular trafficking to a target cell nucleus as compared to an AAV lacking the capsid amino acid mutations. In certain embodiments, the capsid amino acid mutations enhance the interactions with target cells and/or the penetration through the mucus gel layer as compared to an AAV lacking the capsid amino acid mutations. In certain embodiments, the capsid amino acid mutations reduce immunogenicity as compared to an AAV lacking the capsid amino acid mutations. In certain embodiments, the AAV comprises AAV serotypes 1, 2, 3, 4, 5, 6, 7, 8, 9, 10, 11, 12, 13, 14, 15, 16, DJ or DJ/8. In certain embodiments, the AAV is AAV serotype 6 (AAV6). In certain embodiments, the AAV is associated externally with the extracellular vesicle or is contained within the extracellular vesicle. In certain embodiments, the extracellular vesicle is a modified extracellular vesicle derived from a cell transduced with an exogenous nucleic acid sequence. In certain embodiments, the extracellular vesicle is a modified extracellular vesicle comprising one or more modified lipids. In certain embodiments, the extracellular vesicle is a modified extracellular vesicle comprising one or more targeting moieties. In certain embodiments, the extracellular vesicle is derived from eukaryotic cells, such as mammalian cells or insect cells.

In another aspect, a device is provided for delivery of the compositions embodied herein, to an oral cavity, nose, pharynx or to a subject in need thereof.

In another aspect, a method of treating a lung disease or disorder or an oropharyngeal disease comprises administering the compositions to a subject in need thereof. In certain embodiments, a lung disease or disorder comprises cystic fibrosis, chronic obstructive pulmonary disease (COPD), lung inflammation, asthma, lung cancer, bronchitis, infections, or allergies.

In another aspect, a method of modulating an immune response for treating an immune related disorder in a subject in need thereof, comprises administering a composition comprising an extracellular vesicle and an adeno-associated virus (AAV) vector, the AAV vector comprising a nucleic acid sequence encoding an immunomodulatory molecule. In certain embodiments, the AAV comprises one or more amino acid mutations in the AAV capsid proteins. In certain embodiments, the one or more capsid amino acid mutations enhance intracellular trafficking to a target cell nucleus as compared to an AAV lacking the capsid amino acid mutations. In certain embodiments, the capsid amino acid mutations enhance the interactions with target cells and/or the penetration through the mucus gel layer as compared to an AAV lacking the capsid amino acid mutations. In certain embodiments, the capsid amino acid mutations reduce immunogenicity as compared to an AAV lacking the capsid amino acid mutations. In certain embodiments, the AAV comprises AAV serotypes 1, 2, 3, 4, 5, 6, 7, 8, 9, 10, 11, 12, 13, 14, 15, 16, DJ or DJ/8. In certain embodiments, the AAV is AAV serotype 6 (AAV6). In certain embodiments, the AAV is associated externally with the extracellular vesicle or is contained within the extracellular vesicle. In certain embodiments, the immunomodulatory molecule comprises thymulin, interferons, chemokines, cytokines, tumor necrosis factor alpha, modulators of checkpoint inhibitors or ligands of checkpoint inhibitors or combinations thereof. In certain embodiments, an immune related disease or disorder is asthma or allergies. In certain embodiments, the composition is formulated for oral, mucosal, cervicovaginal, ocular or nasal delivery. In certain embodiments, the composition is aerosolized for delivery to the airways by inhalation or the oropharyngeal cavity. In certain embodiments, the composition is formulated as a liquid or gel for delivery to mucosal surfaces. In certain embodiments, the composition is formulated as a suppository.

In another aspect an adeno-associated virus (AAV) vector comprises one or more amino acid mutations in the AAV capsid proteins. In certain embodiments, the one or more mutations are located at one or more conserved phosphorylation sites. In certain embodiments, the mutations comprise T492V, S663V and the combination thereof. In certain embodiments, the AAV vector is associated externally with the extracellular vesicle or is contained within the extracellular vesicle.

In another aspect, a composition comprises a modified extracellular vesicle and a virus vector comprising nucleic acid (e.g. a transgene). In certain embodiments, the extracellular vesicle is a modified extracellular vesicle derived from a cell transduced with an exogenous nucleic acid sequence. In certain embodiments, the extracellular vesicle is a modified extracellular vesicle comprising one or more modified lipids. In certain embodiments, the extracellular vesicle is a modified extracellular vesicle comprising one or more targeting moieties. In certain embodiments, the extracellular vesicle is derived from eukaryotic cells, such as mammalian cells or insect cells. In certain embodiments, the virus vector comprises: adenovirus vectors, adeno-associated virus (AAV) vectors, herpes simplex virus vectors, influenza virus vectors, lentivirus vector, retrovirus vectors, alphavirus vectors, flavivirus vectors, rhabdovirus vectors, measles virus vectors, Newcastle disease virus vectors, poxvirus vectors, vaccinia virus vectors, modified Ankara virus vectors, vesicular stomatitis virus vectors, picornavirus vectors, chimeric virus vectors, synthetic virus vectors, respiratory syncytial virus vectors, parainfluenza virus vectors, foamy virus vectors, recombinant viral vectors, mosaic virus vectors, or pseudotyped virus vectors. In certain embodiments, the virus vector is an AAV vector. In certain embodiments, the AAV vector comprises one or more AAV capsid proteins comprising modified amino acid sequences. In certain embodiments, the modified amino acid capsid proteins comprise one or more insertions, deletions, or substitutions of one or more amino acids. In certain embodiments, the one or more capsid amino acid mutations enhance intracellular trafficking to a target cell nucleus as compared to an AAV lacking the capsid amino acid mutations. In certain embodiments, the capsid amino acid mutations enhance the interactions with target cells and/or the penetration through the mucus gel layer as compared to an AAV lacking the capsid amino acid mutations. In certain embodiments, the capsid amino acid mutations reduce immunogenicity as compared to an AAV lacking the capsid amino acid mutations. In certain embodiments, the AAV comprises AAV serotypes 1, 2, 3, 4, 5, 6, 7, 8, 9, 10, 11, 12, 13, 14, 15, 16, DJ or DJ/8. In certain embodiments, the AAV is AAV serotype 6 (AAV6).

In another aspect, a nucleic acid-based vaccine comprises an extracellular vesicle and a virus vector wherein the virus vector comprises at least one nucleic acid molecule (e.g. transgene). In certain embodiments, the virus vector encodes an immunogenic/antigenic molecule. In certain embodiments, an adjuvant is provided.

### Definitions

The terminology used herein is for the purpose of describing particular embodiments only and is not intended to be limiting of the disclosure. Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used in this specification and the appended claims, the term “or” is generally employed in its sense including “and/or” unless the content clearly dictates otherwise.

The term “about” is used herein to mean a value that is ±10% of the recited value.

The term “AAV” refers to adeno-associated virus and may be used to refer to the naturally occurring wild-type virus itself or derivatives thereof. The term covers all subtypes, serotypes and pseudotypes, and both naturally occurring and recombinant forms, except where required otherwise. The AAV genome is built of single stranded DNA and comprises inverted terminal repeats (ITRs) at both ends of the DNA strand, and two open reading frames: rep and cap, encoding replication and capsid proteins, respectively. A foreign polynucleotide can replace the native rep and cap genes. AAVs can be made with a variety of different serotype capsids which have varying transduction profiles or as used herein, “tropism” for different tissue types. As used herein, the term “serotype” refers to an AAV which is identified by and distinguished from other AAVs based on capsid protein reactivity with defined antisera, e.g., AAV serotypes 1, 2, 3, 4, 5, 6, 7, 8, 9, 10, 11, 12, 13, 14, 15, 16, DJ or DJ/8. For example, serotype AAV2 is used to refer to an AAV which contains capsid proteins encoded from the cap gene of AAV2 and a genome containing 5′ and 3′ ITR sequences from the same AAV2 serotype. Pseudotyped AAV as refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped rAAV are produced using standard techniques described in the art. In certain embodiments, the AAVs comprise mutations in the capsid proteins. These mutations comprise one or more amino acid changes at one or multiple locations of the capsid proteins.

As used herein, by “administering” is meant a method of giving a dosage of a composition described herein (e.g., an EVAAV and/or a pharmaceutical composition thereof) to a subject. The compositions utilized in the methods described herein can be administered by any suitable route, including, for example, by inhalation, nebulization, aerosolization, intranasally, intratracheally, intrabronchially, orally, parenterally (e.g., intravenously, subcutaneously, or intramuscularly), orally, nasally, rectally, topically, or buccally. In some embodiments, a composition described herein is administered in aerosolized particles intratracheally and/or intrabronchially using an atomizer sprayer (e.g., with a MADgic® laryngo-tracheal mucosal atomization device).

The term “amino acid” as used herein refers to naturally occurring and synthetic α, β, γ, and δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. When the term amino acid is used, it is considered to be a specific and independent disclosure of each of the esters of α, β, γ, and δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

As used herein, a “bioactive” or “biologically active” molecule, substance or agent for use herein may comprise a drug active recognizable by one skilled in the art of medicinal chemistry. Such bioactive substances for use herein include, but are not limited to, antibodies and antibody cocktails, nucleic acids, proteins, oligonucleotides including messenger-RNA (mRNA), antidiabetic agents, glucose elevating agents, thyroid hormones, thyroid drugs, parathyroid drugs, vitamins, antihyperlipidemic agents, cardiac drugs, respiratory drugs, nasal decongestants, gastrointestinal drugs, amphetamines, anorexiants, antirheumatic agents, anti-gout agents, migraine drugs, sedatives, hypnotics, antianxiety drugs, anticonvulsants, antidepressants, antipsychotic agents, psychotherapeutic drugs, antimicrobials, antifungals, sulfonamides, antimalaria drugs, antituberculotic drugs, amebicides, antiviral agents, anti-infectives, leprostatics, antihelmintics, antihistamines, antimetabolites, anticholinergics, steroidal anti-inflammatories, anesthetics, antiplatelet drugs, NSAIDs, ace inhibitors, calcium channel blockers, alpha-blockers, muscle relaxers, antihypertensives, vasodilators, diuretics, antiemetics, sex hormones, pituitary hormones, analgesics, uterine hormones, chemotherapeutics, immune checkpoint inhibitors, cytokines, chemokines adrenal steroid inhibitors and the like.

As used herein, a “biomolecule” takes on its ordinary and broad meaning of naturally occurring, or synthetic macromolecules that are associated with a biochemical purpose in an organism. Biomolecules herein may be attached to the outer surface of extracellular vesicles or contained within the extracellular vesicles, and such biomolecules may include nucleotides, nucleosides, oligonucleotides, DNA, RNA, hybridization probes, amino acids, polypeptides, proteins and fragments thereof, and antibodies and fragments thereof.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly used CMV promoter) also comprise enhancer sequences. Enhancers can be located upstream, within, or downstream of coding sequences.

As used herein, the term “extracellular vesicles,” or “EV” for short (“EVs” for plural), refers to cellular-derived lipid encased particles secreted by cells of multicellular organisms into external environments to achieve cellular homeostasis and/or for cell-to-cell communication. Further, the term is meant to include the entire family of extracellular vesicles, including the recently discovered exomeres (extracellular nanoparticles having no apparent biological function), along with engineered or modified extracellular vesicles and mimetic nanovesicles. Natural EVs can be classified into three main types. Namely, apoptotic bodies (500-2000 nm in size), microvesicles (50-1000 nm in size), and exosomes (30-200 nm in size). Besides these size differentiations, the types of vesicles are also differentiated by biogenesis, release pathways, content and function. Exosomes formed by an endosomal route are typically 30-150 nm in diameter. There is no apparent limit as to the type or source of EVs for loading with bioactive substances and formulation into compositions in accordance with the present disclosure. EVs for use herein include, but are not limited to, EVs obtained from plasma, urine, semen, saliva, bronchial fluid, cerebral spinal fluid, breast milk, serum, amniotic fluid, synovial fluid, vaginal secretions, tears, lymph, bile, and gastric acid. In various embodiments, EV's are obtained from producer cells such as for example, stem cells, for example, from mesenchymal stem cells (MSCs). In various embodiments, the EVs are obtained from mammalian producer cells and cell lines, e.g. HEK293 cells. Further, any method of isolation presently known or developed in the future may be used. Current methods for isolating EVs include ultracentrifugation, density gradient methods, size exclusion methods, immunoaffinity capture, and precipitation. For a comprehensive review, see L. M. Doyle, et al., “Overview of Extracellular Vesicles, Their Origin, Composition, Purpose, and Methods for Exosome Isolation and Analysis,” Cells, 8, 727 (2019).

As herein a “gene therapy vector” is any molecule or composition that has the ability to carry a heterologous nucleic acid sequence into a suitable host cell where synthesis of the encoded protein takes place. Typically, a gene therapy vector is a nucleic acid molecule that has been engineered, using recombinant DNA techniques that are known in the art, to incorporate the heterologous nucleic acid sequence. Desirably, the gene therapy vector is comprised of DNA. Examples of gene therapy vectors include viral vectors. However, gene therapy vectors that are not based on nucleic acids, such as liposomes, are also known and used in the art. The gene therapy vector can be integrated into the host cell genome or can be present in the host cell in the form of an episome. In certain embodiments, a gene therapy vector is a virus vector, such as, for example, AAV.

The term “modified,” when used in the context of EVs described herein, refers to an alteration or engineering of an EV, e.g., exosome and/or its producer cell, such that the modified EV is different from a naturally occurring EV. In some aspects, a modified EV described herein comprises a membrane that differs in composition of a protein, a lipid, a small molecular, a carbohydrate, etc. compared to the membrane of a naturally occurring EV (e.g., membrane comprises higher density or number of natural exosome proteins and/or membrane comprises proteins that are not naturally found in exosomes (e.g., antigen, adjuvant, and/or immune modulator). In certain aspects, such modifications to the membrane changes the exterior surface of the EV (e.g., surface engineered EV). In certain aspects, such modifications to the membrane changes the lumen of the EV.

As used herein, the term “modulate” includes to “increase” or “decrease” one or more quantifiable parameters, optionally by a defined and/or statistically significant amount. By “increase” or “increasing,” “enhance” or “enhancing,” or “stimulate” or “stimulating,” refers generally to the ability of one or more vesicle-based compositions in accordance with the present disclosure to produce or cause a greater physiological response (i.e., downstream effects) in a cell or in a subject relative to the response caused by either no vesicle-based composition or a control compound. Relevant physiological or cellular responses (in vivo or in vitro) upon administration of vesicle-based compositions will be apparent to persons skilled in the art. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times), including all integers and decimal points in between and above 1 (e.g., 1.5, 1.6, 1.7, 1.8), the amount produced by no vesicle-based composition (the absence of a bioactive agent) or a control compound. The term “reduce” or “inhibit” may relate generally to the ability of one or more vesicle-based compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a disease, illness or condition, such as cancer, inflammation or pain. A “decrease” in a response may be “statistically significant” as compared to the response produced by no vesicle-based composition or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25% 30%, 35%, 40%, 45%, 50%, 55%, 60%, 65%, 70%, 75%, 80%, 85%, 90%, 95%, or 100% decrease, including all integers in between.

The terms “patient” or “individual” or “subject” are used interchangeably herein, and refers to a mammalian subject to be treated, with human patients being preferred. In some cases, the methods of the disclosure find use in experimental animals, in veterinary application, and in the development of animal models for disease, including, but not limited to, rodents including mice, rats, and hamsters, and primates.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In some aspects, the EVs useful in the present disclosure do not carry an antigen on MHC class I or class II molecule (i.e., antigen is not presented on MHC class I or class II molecule) exposed on the surface of the EV.

As used herein, “recombinant adeno-associated virus (AAV)” or “rAAV vector” is meant a recombinantly-produced AAV or AAV particle that comprises a polynucleotide sequence not of AAV origin (e.g., a polynucleotide comprising a transgene, which may be operably linked to one or more enhancer and/or promoters) to be delivered into a cell, either in vivo, ex vivo, or in vitro. Non-naturally occurring (e.g., chimeric) capsids may be used in the rAAVs.

A “therapeutic gene,” “prophylactic gene,” “target polynucleotide,” “transgene,” “gene of interest,” “exogenous gene” and the like generally refer to a gene or genes to be transferred using a vector. Typically, in the context of the present disclosure, such genes are located within the rAAV vector (which vector is flanked by inverted terminal repeat (ITR) regions and thus can be replicated and encapsidated into rAAV particles). Target polynucleotides can be used in this disclosure to generate rAAV vectors for a number of different applications. Such polynucleotides include, but are not limited to: (i) polynucleotides encoding proteins useful in other forms of gene therapy to relieve deficiencies caused by missing, defective or sub-optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators such as cytokines; (v) polynucleotides that can make recipient cells susceptible to specific drugs, such as the herpes virus thymidine kinase gene; (vi) polynucleotides for cancer therapy, such as E1A tumor suppressor genes or p53 tumor suppressor genes for the treatment of various cancers; and (vii) polynucleotides for gene editing (e.g., CRISPR). To effect expression of the transgene in a recipient host cell, it is in one embodiment operably linked to a promoter, either its own or a heterologous promoter. A large number of suitable promoters are known in the art, the choice of which depends on the desired level of expression of the target polynucleotide, whether one desires constitutive expression, inducible expression, cell-specific or tissue-specific expression, etc. The rAAV vector may also contain a selectable marker. Exemplary transgenes include, without limitation, cystic fibrosis transmembrane conductance regulator (CFTR) or derivatives thereof (e.g., a CFTRAR minigene; see, e.g., Ostedgaard et al. Proc. Natl. Acad. Sci. USA 108(7):2921-6, 2011, which is incorporated by reference herein in its entirety), α-antitrypsin, β-globin, γ-globin, tyrosine hydroxylase, glucocerebrosidase, aryl sulfatase A, factor VIII, dystrophin, erythropoietin, alpha 1-antitrypsin, surfactant protein SP-D, SP-A or SP-C, erythropoietin, growth factors, or a cytokine, e.g., IFN-alpha, IFNγ, TNF, IL-1, IL-17, or IL-6, or a prophylactic protein that is an antigen such as viral, bacterial, tumor or fungal antigen, or a neutralizing antibody or a fragment thereof that targets an epitope of an antigen such as one from a human respiratory virus, e.g., influenza virus or RSV including but not limited to HBoV protein, influenza virus protein, RSV protein, or SARS protein.

As used herein, the term “subject” or the phrase “a subject in need thereof” refers to any human or non-human animal requiring or desirous of a pharmacological change. For example, a subject in need thereof may be a human patient clinically diagnosed with a disease, such as, for example, cystic fibrosis, and is thus in need of, or is at least desirous of receiving some sort of treatment.

As used herein, the term “treatment” of a subject (e.g., a mammal, such as a human) or a cell, is any type of intervention used in an attempt to alter the natural course of the subject or cell. Treatment includes, but is not limited to, administration of a vesicle-based composition in accordance with the present disclosure and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

As used herein, the term “therapeutically effective amount” refers to a minimum dosage of a composition in accordance with the present disclosure that provides a desired effect. Therefore, a therapeutically effective amount varies by subject, dosage form/concentration, and results desired. For example, a therapeutically effective amount of an inhalable liquid or aerosol to treat a CF or COPD, might be on the order of micrograms or milligrams per day. In other examples, a therapeutically effective amount of a nasal spray composition, might be on the order of 3 sprays per day per nostril, with each spray about 0.1 mL.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D demonstrate that EVAAV6 prepared in HEK293 cells and harvested from the cell culture supernatant exhibits internal and external association of AAV6 with EV. (FIG. 1A) Western blot analysis of AAV6, EV and EVAAV6. Bands for VP1, 2 and 3 of AAV6 capsid and for syntenin-1 (ST1) and CD9 of EV are shown. (FIG. 1B) ExoView analysis demonstrating that exosomes are a primary population similarly in EV and EVAAV6. (FIG. 1C) Hydrodynamic diameters of AAV6, EV and EVAAV6 measured by DLS. (FIG. 1D) Representative transmission electron micrographs of AAV6, EV, EVAAV6 and EV+AAV6. Scale bar=50 nm.

FIGS. 2A and 2B demonstrate that AAV6 exhibits significantly greater diffusion rates compared to AAV1 in multiple independent sputum samples freshly expectorated by patients with MOLDs. (FIG. 2A) Representative AAV trajectories in different sputum samples. Scale bar=1 μm. (FIG. 2B) Box-and-whisker plots of MSD of gene vectors at t=1 s in sputum samples collected from 8 different CF patients. *p<0.05 (Student's t-test).

FIGS. 3A and 3B demonstrate that EVAAV6 exhibits efficient penetration through human airway mucus and enhanced transduction of human bronchial epithelial (HBE) cell line. (FIG. 3A) Median MSD values of EV and EVAAV6 in sputum samples spontaneously expectorated by CF patients. MSD is a square of distance traveled by an individual particulate matter within a predetermined time interval (i.e., time scale; τ=1 s) and thus is directly proportional to the particle diffusion rate. The upper and lower red dashed lines indicate theoretical MSD values of EV in PBS calculated by the Stoke-Einstein equation and MSD values of AAV6 previously measured in CF sputum, respectively. n.s.: no significance (two-tailed Student's t-test) (FIG. 3B) Luciferase activity measured in lysates of HBE cells treated with EV, EVAAV6 or EV+AAV6. n.s.: no significance, ****p<0.0001 (one-way ANOVA).

FIGS. 4A-4E demonstrate that EVAAV6 provides widespread and enhanced reporter transgene expression in mouse lungs following intratracheal administration. (FIG. 4A) Representative confocal images demonstrating reporter transgene expression throughout the whole left lung lobes of mice intratracheally treated with normal saline, AAV6, EVAAV6 or EV+AAV6. Green: YFP, Blue: nuclei. Image-based quantification of (FIG. 4B) coverage and (FIG. 4C) intensity of YFP transgene expression in whole lung lobes treated with normal saline, AAV6, EVAAV6 or EV+AAV6. (FIGS. 4D-4E) Luciferase activity measured in lysates of the whole mouse lungs treated with normal saline, AAV6, EVAAV6 or EV+AAV6. Mice receiving EVAAV6 or EV+AAV6 were treated at a (FIG. 4D) lower and (FIG. 4E) higher EV-to-AAV6 ratios. n.s.: no significance, **p<0.01, ***p<0.001, ****p<0.0001 (one-way ANOVA).

FIGS. 5A-5C demonstrate that EVAAV6 provides enhanced reporter transgene expression in mucus-secreting ALI cultures of primary wild-type (WT) or cystic fibrosis (CF) HBE cells following apical administration. (FIG. 5A) Representative confocal images demonstrating reporter transgene expression in ALI cultures of primary WT or CF HBE cells treated with normal saline, AAV6, EVAAV6 or EV+AAV6. Green: YFP, Blue: nuclei. Image-based quantification of (FIG. 5B) coverage and (FIG. 5C) intensity of YFP transgene expression in the ALI cultures treated with normal saline, AAV6, EVAAV6 or EV+AAV6. n.s.: no significance, ***p<0.001, ****p<0.0001 (two-way ANOVA).

FIG. 6 is a graph demonstrating that capsid-optimized AAV6 mutants exhibit greater diffusion rates in multiple independent sputum samples compared to wild-type AAV6. Four tested mutant AAV6 (mAAV6) exhibit greater diffusion rates, as indicated by averages of median MSD values, compared to those of wild-type AAV6 (red dashed line).

FIGS. 7A-7C demonstrate that thymulin gene therapy with mucus-permeable synthetic nanoparticles provides comprehensive therapeutic benefits in the lungs of OVA-induced allergic asthma model. CTRL-SAL: sensitized/challenged with saline and treated with saline (healthy control); OVA-SAL: sensitized/challenged with OVA and treated with saline (disease/untreated control); OVA-THY: sensitized/challenged with OVA and treated with thymulin gene therapy. (FIG. 7A) Representative photomicrographs of histologically stained with hematoxylin and eosin (H&E). Scale bars denote 200=m. (FIG. 7B) Levels of Th-2 cytokines and pro-fibrotic mediators in BALF. (FIG. 7C) Measurement of AHR (i.e., lung mechanics). *p<0.05 compared to the CTRL-SAL group; #p<0.05 compared to the OVA-SAL group.

FIG. 8 is a schematic representation of specific aims and respective experiments outlined in the examples section.

FIGS. 9A and 9B demonstrate that EV and EVAAV6 exhibit similar particle sizes as measured by Spectradyne nCS1™ particle size analyzer. The average particle diameters and concentrations, respectively, for (FIG. 9A) EV are 63.0 nm and 6.15×10¹¹/mL and for (FIG. 9B) EVAAV6 are 63.1 nm and 9.47×10¹¹/mL. The measured concentrations have been used to match the EV concentrations in EV+AAV6 and EVAAV6 samples for in vitro and in vivo comparisons.

FIG. 10 demonstrates that EVAAV6 with a higher EV-to-AAV6 ratio exhibit greater transgene expression on average following intratracheal administration, but the difference is not statistically significant. The EV-to-AAV6 ratios of EVAAV6 with low and high EV contents are 2.17 and 18.5, respectively. n.s. no significance (two-sided Student's I-test).

FIG. 11 shows luciferase activity measured in lysates of the whole mouse lungs treated with EVAAV6 or mAAV6 vectors piggybacked on EVs (EVmAAV6). N=6 animals per group, *p<0.05 (two-sided Student's t-test).

### DETAILED DESCRIPTION

The disclosure involves preparation and use of extracellular vesicle (EV)-associated adeno-associated virus (AAV) for inhaled lung-directed gene therapy applications. It was discovered that EV-associated AAV serotype 6 (EVAAV6), produced during AAV preparation in HEK293 cells, provides markedly and significantly greater transgene expression in human primary airway cells and in mouse lungs (following inhaled administration) compared to AAV6 which has been shown greater performances compared to other widely used AAV serotypes (e.g., AAV1, 2 and 5) in transducing lung airway cells. This enhancement is attributed to the ability of EV to promote mucus penetration and cellular transduction of AAV6. The approach is likely applicable/beneficial to other AAV serotypes or virus vectors incapable of penetrating the airway mucus barrier, thereby broadening the AAV serotypes and other virus vectors that can be utilized for inhaled gene therapy applications.

### Gene Therapy Vectors

Gene delivery vectors within the scope of the disclosure include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with ether molecules such as DNA-anti-DNA antibody-cationic lipid (DOTMA/DOPE) complexes. Exemplary viral gene delivery vectors are desorbed below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch.

In one embodiment, the gene therapy vector is a viral vector. Suitable viral vectors include, for example, retroviral vectors, lentivirus vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley 4 Sons, New York, N.Y. (1994).

Retroviral Vectors: Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

Lentiviruses: Lentiviruses are derived from a family of retroviruses that Include human Immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., Meth. Mol. Med., 69:427 (2002)).

Adenoviral Vectors: Adenoviral vectors may be rendered replication-incompetent by deleting the early (E1A and E1B) genes responsible for viral gene expression from the genome and are stably maintained into the host cells m an extrachromosomal form. These vectors have the ability to transfect both replicating and nonreplicating cells. Adenoviral vectors have been shown to result in transient expression of therapeutic genes in vivo, peaking at 7 days and lasting approximately 4 weeks. The duration of nucleic acid (transgene) expression may be improved in systems utilizing neural specific promoters. In addition, adenoviral vectors can be produced at very high titers, allowing efficient gene therapy with small volumes of virus.

Adeno-Associated Virus Vectors: Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and non replicating cells and are believed to be nonpathogenic to humans.

Exemplary AAV Vectors: In certain embodiments, the disclosure provides an adeno-associated virus (AAV) vector which comprises a nucleic acid sequence a therapeutic molecule. Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78. Rep68, Rep62, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see. e.g., Im et al., Cell. 51:447 (1990)). These proteins also regulate transcription from endogenous AAV promoters and promoters within helper viruses (see, e.g., Pereira et al., J Virol., 71:1079 (1997). The other Rep proteins modify the function of Rep78 and Rep68. The cap genes encode the capsid proteins VP1, VP2, and VP3. The cap genes are transcribed from the p40 promoter.

The AAV vector may be generated using any AAV serotype known in the art. Several AAV serotypes and over 100 AAV variants have been isolated from adenovirus stocks or from human or nonhuman primate tissues (reviewed in. e.g., Wu et al., Molecular Therapy. 14(3): 316 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent and replicate and assemble by practically identical mechanisms. AAV serotypes 1-5 and 7-9 are defined as “true” serotypes, in that they do not efficiently cross-read with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy due to its lack of pathogenicity, wide range of infectivity, and ability to establish long-term transgene expression (see, e.g., Carter, Hum. Gone Ther., 15:541 (2005). Genome sequences of various AAV serotypes and comparisons thereof are disclosed in, for example, GenBank Accession numbers U89790, J01901, AF043303, and AF085716: Chiorini et al., J. Virol., 71:6823 (1997); Srivastava et al., J. Virol., 45:565 (1983); Chiorini et al., J. Virol. 23:1309 (1999); Rutledge et al., J. Virol., 22-309 (1998); and Wu et al., J. Virol., 24:8635 (2000)).

AAV rep and ITR sequences are particularly conserved across most AAV serotypes. For example, the Rep78 proteins of AAV2, AAV3A, AAV3B, AAV4, and AAV6 are reportedly about 89-93% identical (see Bantel-Schaal et al., J Virol., 73(2):939 (1999)). It has been reported that AAV serotypes 2, 3A, 3B. and 6 share about 82% total nucleotide sequence identity at the genome level (Bantel-Schaal et al., supra). Moreover, the rep sequences and ITRs of many AAV serotypes are known to efficiently cross-complement (e.g., functionally substitute) corresponding sequences from other serotypes during production of AAV particles in eukaryotic cells, such as mammalian cells or insect cells.

Generally, the cap proteins, which determine the cellular tropicity of the AAV particle, and related cap protein-encoding sequences, are significantly less conserved than Rep genes across different AAV serotypes. In view of the ability Rep and ITR sequences to cross-complement corresponding sequences of other serotypes, the AAV vector can comprise a mixture of serotypes and thereby be a ‘chimeric’ or ‘pseudotyped’ AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551: Flotte, Mol. Ther., 12(1):1 (2006); Gao et al., J. Virol., 78:6381 (2004); Gao et al., Proc. Natl. Acad. Sci. USA, 23:11854 (2002); De et al., Mol. Ther., 12:67 (2006); and Gao et al., Mol. Ther., 12:77 (2006). In certain embodiments, the AAV comprises one or more mutations in the capsid protein at one or more locations in the capsid protein.

In one embodiment, the AAV vector is generated using an AAV that infects humans (e.g., AAV6). Alternatively, the AAV vector is generated using an AAV that infects non-human primates, such as, for example, the great apes (e.g., chimpanzees). Old World monkeys (e.g., macaques) and New World monkeys (e.g., marmosets). In one embodiment, the AAV vector is generated using an AAV that infects a non-human primate pseudotyped with an AAV that infects humans. Examples of such pseudotyped AAV vectors are disclosed in, e.g., Cearley et al., Molecular Therapy. 12:528 (2006).

In addition to the nucleic acid sequence encoding a therapeutic gene, the AAV vector may comprise expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology. Methods in Enzymology. Vol. 185. Academic Press. San Diego. Calif. (1990).

A large number of promoters, including constitutive, inducible, and repressive promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., J2:3346 (1996)), the T-REX™ system (Invitrogen, Carlsbad, Calif.), LACSWITCH™ System (Stratagene, San Diego, Calif.), and the Cre-ERT tamoxifen inducible recombinase system (Indra et al., Nuc. Acid. Res., 22:4324 (1999); Nuc. Acid. Res., 28:e99 (2000); U.S. Pat. No. 7,112,715; and Kramer & Fuseenegger. Methods Mol. Bid., 308:123 (2005)).

Typically, AAV vectors are produced using well characterized plasmids. For example, human embryonic kidney 293T cells are transfected with one of the transgene specific plasmids and another plasmid containing the adenovirus helper and AAV rep and cap genes. After 72 hours, the cells are harvested, and the vector is released from the cells by five freeze/thaw cycles. Subsequent centrifugation and berizonase treatment remove cellular debris and unencapsidated DNA. Iodixanol gradients and ion exchange columns may be used to further purify each AAV vector. Next the purified vector is concentrated by a size exclusion centrifuge spin column to the required concentration. Finally, the buffer is exchanged to create the final vector products formulated (for example) in 1.times. phosphate buffered saline. The viral titers may be measured by TaqMan™ real-time PCR and the viral purity may be assessed by SDS-PAGE.

### Modified AAV Capsid Proteins and Virus Capsids and Virus Vectors

The present disclosure provides AAV capsid proteins comprising at least one mutation (i.e., a modification, which can be a substitution or an insertion or a deletion) in the amino acid sequence and virus capsids and virus vectors comprising the modified AAV capsid protein. The mutated AAV capsid proteins, and the nucleic acids encoding them, are not found in nature (i.e., are non-natural) and have neither the sequence of the wild-type sequences found in nature nor the function of those sequences. In certain embodiments, the modifications at the one or more amino acid positions described can confer one or more desirable properties to virus vectors comprising the modified AAV capsid protein including without limitation: (i) enhancing intracellular trafficking to a target cell nucleus; (ii) enhancing the interactions with target cells; (iii) enhancing penetration through the mucus gel layer.

In certain embodiments, the modified AAV capsid protein of the disclosure comprises one or more mutations (i.e., modifications) in the amino acid sequence of the native, e.g., AAV6 capsid protein or the corresponding amino acid residue(s) of a capsid protein from another AAV serotype, including but not limited to AAV2, AAV3, AAV6, AAV7, AAV8, AAV9, AAV10, etc. The amino acid positions in other AAV serotypes or modified AAV capsids that “correspond to” these positions in the native AAV6 capsid protein will be apparent to those skilled in the art and can be readily determined using sequence alignment techniques and/or crystal structure analysis (Padron et al. (2005) J. Virol. 79:5047-58).

Those skilled in the art will appreciate that for some AAV capsid proteins the corresponding modification will be an insertion and/or a substitution, depending on whether the corresponding amino acid positions are partially or completely present in the virus or, alternatively, are completely absent. Likewise, when modifying AAV, the specific amino acid position(s) may be different than the position in AAV6.

As used herein, a “mutation” or “modification” in an amino acid sequence includes substitutions, insertions and/or deletions, each of which can involve one, two, three, four, five, six, seven, eight, nine, ten or more amino acids. In certain embodiments, the modification is a substitution. In other embodiments, the modification is an insertion (e.g., of a single amino acid residue between two amino acid residues in an amino acid sequence). In certain embodiments, the modification is a deletion. In certain embodiments, the modification is a modified amino acid.

It is to be understood that the substitutions and insertions described in the AAV capsid proteins of this disclosure can include substitutions and/or insertions with conservative amino acid residues. Such conservative substitutions are well known in the art and include, e.g., nonpolar amino acids Gly, Ala, Val, Leu, Ile, Met, Phe, Trp and Pro can be substituted for one another; polar amino acids Ser, Thr, Cys, Tyr, Asn and Gln can be substituted for one another; negatively charged amino acids Asp and Glu can be substituted for one another; and positively charged amino acids Lys, Arg and His can be substituted for one another, in any combination.

The present disclosure also provides an AAV capsid comprising one or more mutations as well as a virus vector comprising one or more mutations.

In certain embodiments, recombinant AAV genomes comprise a transgene and one or more AAV ITRs flanking a nucleic acid molecule. AAV DNA in the rAAV genomes may be from any AAV serotype for which a recombinant virus can be derived including, but not limited to, AAV serotypes 1, 2, 3, 4, 5, 6, 7, 8, 9, 10, 11, 12, 13, 14, 15, 16, DJ or DJ/8. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014).

Techniques to produce rAAV particles, in which an AAV genome to be packaged, rep and cap genes, and helper virus functions are provided, are standard in the art. Production of rAAV requires that the following components are present within a single cell (denoted herein as a packaging cell): a rAAV genome, AAV rep and cap genes separate from the rAAV genome, and helper virus functions. The AAV rep and cap genes may be from any AAV serotype for which recombinant virus can be derived and may be from a different AAV serotype than the rAAV genome ITRs, including, but not limited to, AAV serotypes 1, 2, 3, 4, 5, 6, 7, 8, 9, 10, 11, 12, 13, 14, 15, 16, DJ or DJ/8. Production of pseudotyped rAAV is disclosed in, for example, WO 01/83692 which is incorporated by reference herein in its entirety.

Methods of generating a packaging cell comprise creating a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79: 2077-2081), addition of synthetic linkers containing restriction endonuclease cleavage sites (Laughlin et al., 1983, Gene, 23: 65-73) or by direct, blunt-end ligation (Senapathy & Carter, 1984, J. Biol. Chem., 259: 4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

### Extracellular Vesicles

Also provided herein are extracellular vesicles (EVs) for delivery of virus vectors, e.g. AAV. Exemplary extracellular vesicles may include but are not limited to exosomes. However, the term “extracellular vesicles” should be interpreted to include all nanometer-scale lipid vesicles that are secreted by cells such as secreted vesicles formed from lysosomes.

EVs are cell-derived vesicles with a closed double-layer membrane structure. According to their size and density, EVs mainly include exosomes (30-150 nm), micro vesicles (MVs) (100-1000 nm), and apoptotic bodies or cancer related oncosomes (1-10 μm). EVs are able to carry various molecules, such as proteins, lipids and RNAs on their surface as well as within their lumen. The EV and exosomal surface proteins can mediate organ-specific homing of circulating EVs.

EVs are produced by many different types of cells including immune cells such as B lymphocytes, T lymphocytes, dendritic cells (DCs) and most cells. EVs are also produced, for example, by glioma cells, platelets, reticulocytes, neurons, intestinal epithelial cells, and tumor cells. EVs for use in the disclosed compositions and methods can be derived from any suitable cells, including the cells identified above. EVs have also been isolated from physiological fluids, such as plasma, urine, amniotic fluid and malignant effusions. Non-limiting examples of suitable EVs producing cells for mass production include dendritic cells (e.g., immature dendritic cell), Human Embryonic Kidney 293 (HEK) cells, 293T cells, Chinese hamster ovary (CHO) cells, and human ESC-derived mesenchymal stem cells. Other cell sources are useful for providing exosomes useful for bioactive loading in accordance with the present disclosure. These include, stem cells, cancer cell, myeloid cells, brain cells, bioreactors, and exosomes from plant, fungal or bacterial cells. Also, depending on the application, either for pain management, inflammation, neurodegenerative, autoimmune, cancer or recreational applications, the source of exosomes may vary as well as the desired cells to target will vary depending on the application and disease.

EVs can also be obtained from any autologous patient-derived, heterologous haplotype-matched or heterologous stem cells so to reduce or avoid the generation of an immune response in a patient to whom the EVs are delivered. Any EV-producing cell can be used for this purpose.

In certain embodiments, the extracellular vesicle is an EV mimetic-nanovesicles (M-NVs). M-NVs are a type of artificial EVs which can be generated from all cell types with comparable characteristics as EVs for alternative therapeutic modality.

EVs produced from cells can be collected from the culture medium by any suitable method. Typically, a preparation of EVs can be prepared from cell culture or tissue supernatant by centrifugation, filtration or combinations of these methods. For example, EVs can be prepared by differential centrifugation, that is low speed (<20000 g) centrifugation to pellet larger particles followed by high speed (>100000 g) centrifugation to pellet EVs, size filtration with appropriate filters (for example, 0.22 μm filter), gradient ultracentrifugation (for example, with sucrose gradient) or a combination of these methods.

The compositions embodied herein, e.g., EV-virus vector (e.g. AAV), may be administered to a subject by any suitable means. Administration may be topical (including ophthalmic and to mucus membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Vesicle-based compositions for topical administration may include transdermal patches, ointments, lotions, creams, gels, drops, suppositories, sprays, liquids and powders. Conventional pharmaceutically acceptable carriers, aqueous, powder or oily bases, thickeners and the like, may be necessary or desirable. In certain embodiments, delivery is via the intranasal or oral route. A physician will be able to determine the required route of administration for each particular patient.

In certain embodiments, the EVAAVs are delivered as a composition. The composition may be formulated for inhalation, by nebulization, or by aerosolization, or is intranasal, intratracheal, intrabronchial, oral, intravenous, subcutaneous, and/or intramuscular administration. In addition, the compositions may be administered via parenteral intracerebral, intravascular (including intravenous), subcutaneous, or transdermal administration. Compositions for parenteral administration may include sterile aqueous solutions which may also contain buffers, diluents and other suitable additives. The EVs may be formulated in a pharmaceutical composition, which may include pharmaceutically acceptable carriers, thickeners, diluents, buffers, preservatives, and other pharmaceutically acceptable carriers or excipients and the like in addition to the EVs.

In certain embodiments, the EVs are loaded with a desired bioactive substance, e.g., a therapeutic, medicinal, homeopathic agent, and the resulting loaded EVAAVs are targeted to the desired location in the subject by composition dosage form and administration route where the vesicles can release their contents to the target of interest. In certain embodiments, the bioactive molecule is a toxin, toxoid, or a non-toxic mutant of a toxin.

In some embodiments, the biologically active molecule is an activator for a positive co-stimulatory molecule or an activator for a binding partner of a positive co-stimulatory molecule. In some embodiments, the positive co-stimulatory molecule is a TNF receptor superfamily member. In some embodiments, the TNF receptor superfamily member is selected from the group consisting of: CD120a, CD120b, CD18, OX40, CD40, Fas receptor, M68, CD27, CD30, 4-1BB, TRAILR1, TRAILR2, TRAILR3, TRAILR4, RANK, OCIF, TWEAK receptor, TACI, BAFF receptor, ATAR, CD271, CD269, AITR, TROY, CD358, TRAMP, and XEDAR. In some embodiments, the activator for a positive co-stimulatory molecule is a TNF superfamily member. In some embodiments, the TNF superfamily member is selected from the group consisting of: TNFα, TNF-C, OX40L, CD40L, FasL, LIGHT, TL1A, CD27L, Siva, CD153, 4-1BB ligand, TRAIL, RANKL, TWEAK, APRIL, BAFF, CAMLG, NGF, BDNF, NT-3, NT-4, GITR ligand, and EDA-2. In some embodiments, the positive co-stimulatory molecule is a CD28-superfamily co-stimulatory molecule. In some embodiments, the CD28-superfamily co-stimulatory molecule is ICOS or CD28. In some embodiments, the activator for a positive co-stimulatory molecule is ICOSL, CD80, or CD86.

In other embodiments, the cytokine is selected from the group consisting of: IL-2, IL-7, IL-10, IL-12, and IL-15. In some embodiments, the protein comprises a T-cell receptor (TCR), a T-cell co-receptor, a major histocompatibility complex (MHC), a human leukocyte antigen (HLA), or a derivative thereof. In some embodiments, the protein comprises a tumor antigen. In some embodiments, the tumor antigen is selected from the group consisting of: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen, MART-1 gp100, and TNF-related apoptosis-inducing ligand.

Modified EVs: In certain embodiments, the EVs are modified EVs. A desired modification of an EV can be introduced by transducing a eukaryotic cell line (e.g. mammalian cell line), e.g., HEK293 cells with a desired polynucleotide. For example, a desired polynucleotide can encode a targeting or tropism moiety which specifically targets the EVAAVs to a desired organ, tissue or cell. Non-limiting examples of tropism moieties that can be used with the present disclosure include those that can bind to a marker expressed specifically on an alveolar epithelial cell (AEC), lung fibroblasts, pulmonary artery endothelial cells (PAEC), pulmonary artery smooth muscle cells (PASMC) or respiratory epithelial cells. Unless indicated otherwise, the term “targeting moiety,” as used herein, encompasses tropism moieties. The targeting moiety can be a biological molecule, such as a protein, a peptide, a lipid, or a carbohydrate, or a synthetic molecule. For example, the targeting moiety can be an affinity ligand (e.g., antibody, VHH domain, phage display peptide, fibronectin domain, camelid, aptamer, VNAR), a synthetic polymer (e.g., PEG), a natural ligand/molecule (e.g., CD40L, albumin, CD47, CD24, CD55, CD59), a recombinant protein, but not limited thereto.

In certain aspects, the targeting moiety, is displayed on the surface of EVs (e.g., exosomes). The targeting moiety can be displayed on the EV surface by being fused to a scaffold protein (e.g., as a genetically encoded fusion molecule). In some aspects, the targeting moiety can be displayed on the EV surface by chemical reaction attaching targeting moiety to an EV surface molecule. A non-limiting example is PEGylation. In some aspects, EVs disclosed can further comprise a targeting moiety, in addition to an antigen, adjuvant, or immune modulator.

In certain embodiments, an engineered extracellular vesicle (eEV), comprises an extracellular vesicle isolated from a biological cell with at least one lipid incorporated into the membrane thereof.

In this embodiment the lipid may be a synthetic lipid or an exogenous lipid/non-native lipid or a combination thereof. A synthetic lipid comprises 1-palmitoyl-2-oleoyl-sn-glycero-3-phosphocholine (POPC), 1-palmitoyl-2-oleoyl-sn-glycero-3-phosphoglycerol (POPG), dipalmitoylphosphatidylcholine (DPPC), or 1,2-dipalmitoyl-sn-glycero-3-phosphocholine (DOTAP) or a combination thereof. Also, the biological cell may be associated with a pathophysiological condition. In one aspect the pathophysiological condition may be a cancer. In another aspect the pathophysiological condition is cystic fibrosis, COPD, asthma, tuberculosis, or an infection, such as by a virus, bacteria or parasite. In another aspect the biological cell may be a primary mesenchymal stem cell, an embryonic kidney cell, an embryonic fibroblast cell, an alveolar basal epithelial cell, or a monocytic cell or an immortalized cell-line thereof.

In another embodiment, there is provided a method for preparing an engineered extracellular vesicle, comprising the steps of culturing the biological cell in vitro in a culture medium; isolating the extracellular vesicles from the biological cells; and extruding the isolated extracellular vesicles with the at least one lipid to form the engineered extracellular vesicle.

The extracellular vesicle delivery vehicle can comprise at least one lipid hybridized with a membrane of the extracellular vesicle; a nucleic acid loaded within a core of the extracellular vesicle; and a therapeutic drug complexed with the extracellular vesicle either within or on the surface. The lipid may be a synthetic lipid comprising 1-palmitoyl-2-oleoyl-sn-glycero-3-phosphocholine (POPC), 1-palmitoyl-2-oleoyl-sn-glycero-3-phosphoglycerol (POPG), dipalmitoylphosphatidylcholine (DPPC), or 1,2-dipalmitoyl-sn-glycero-3-phosphocholine (DOTAP) or a combination thereof. Also, the nucleic acid may be a synthetic DNA, a naturally occurring DNA, a synthetic RNA, or a naturally occurring RNA, or fragments thereof. Particularly, the synthetic RNA or naturally occurring RNA may be a small-interfering RNA (siRNA) or a microRNA (miRNA).

Methods for preparing engineered extracellular vesicles (eEV) include isolating extracellular vesicles from a biological cell of interest. The membranes of the extracellular vesicles are hybridized or modified with at least one synthetic lipid, for example, a synthetic hydrated lipid, such as by extrusion, to produce a modified or hybrid engineered extracellular vesicles. Examples of suitable synthetic lipids are, but not limited to, 1-Palmitoyl-2-oleoyl-sn-glycero-3-phosphocholine (POPC), 1-Palmitoyl-2-oleoyl-sn-glycero-3-phosphoglycerol (POPG), dipalmitoylphosphatidylcholine (DPPC), or 1,2-dipalmitoyl-sn-glycero-3-phosphocholine (DOTAP).

### Delivery

Compositions described herein (e.g., EV-rAAVs, pharmaceutical compositions) may be used in vivo as well as ex vivo. In vivo gene therapy comprises administering the vectors of this disclosure directly to a subject. Pharmaceutical compositions can be supplied as liquid solutions or suspensions, as emulsions, or as solid forms suitable for dissolution or suspension in liquid prior to use. For administration into the respiratory tract, one exemplary mode of administration is by aerosol, using a composition that provides either a solid or liquid aerosol when used with an appropriate aerosolubilizer device. Another some mode of administration into the respiratory tract is using a flexible fiberoptic bronchoscope to instill the vectors. Typically, the viral vectors are in a pharmaceutically suitable pyrogen-free buffer such as Ringer's balanced salt solution (pH 7.4). Although not required, pharmaceutical compositions may optionally be supplied in unit dosage form suitable for administration of a precise amount.

A composition described herein (e.g., EV-rAAVs, pharmaceutical compositions) can be administered by any suitable route, e.g., by inhalation, nebulization, aerosolization, intranasally, intratracheally, intrabronchiially, orally, parenterally (e.g., intravenously, subcutaneously, or intramuscularly), orally, nasally, rectally, topically, or buccally. They can also be administered locally or systemically. In some embodiments, a composition described herein is administered in aerosolized particles intratracheally and/or intrabronchially using an atomizer sprayer (e.g., with a MADgic® laryngo-tracheal mucosal atomization device). In some embodiments, the pharmaceutical composition is administered parentally. In other some embodiments, the pharmaceutical composition is administered systemically. Vectors can also be introduced by way of bioprostheses, including, by way of illustration, vascular grafts (PTFE and dacron), heart valves, intravascular stents, intravascular paving as well as other non-vascular prostheses. General techniques regarding delivery, frequency, composition and dosage ranges of vector solutions are within the skill of the art.

For administration to the upper (nasal) or lower respiratory tract by inhalation, the compositions described herein (e.g., EV-rAAVs, pharmaceutical compositions.) are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the agent may be administered via nose drops, a liquid spray, such as via a plastic bottle atomizer or metered-dose inhaler. Typical of atomizers are the Mistometer (Wintrop) and the Medihaler (Riker).

Administration of the compositions described herein (e.g., EV-rAAVs, pharmaceutical compositions) may be continuous or intermittent, depending, for example, upon the recipient's physiological condition, whether the purpose of the administration is therapeutic or prophylactic, and other factors known to skilled practitioners. The EV-rAAVs or pharmaceutical compositions described herein can be administered once, or multiple times, at the same or at different sites. The administration of the agents of the disclosure may be essentially continuous over a preselected period of time or may be in a series of spaced doses.

The compositions described herein (e.g., rAAVs, pharmaceutical compositions) can be administered in combination with one or more additional therapeutic agent. Any suitable additional therapeutic agent(s) may be used, including standard of care therapies, e.g. for CF. In some embodiments, the one or more additional therapeutic agents includes an antibiotic (e.g., azithromycin (ZITHROMAX®), amoxicillin and clavulanic acid (AUGMENTIN®), cloxacillin and diclocacillin, ticarcillin and clavulanic acid (TIMENTIN®), cephalexin, cefdinir, cefprozil, cefaclor; sulfamethoxazole and trimethoprim (BACTRIM®), erythromycin/sulfisoxazole, erythromycin, clarithromycin, tetracycline, doxycycline, minocycline, tigecycline, vancomycin, imipenem, meripenem, Colistimethate/COLISTIN®, linezolid, ciprofloxacin, levofloxacin, or a combination thereof), a mucus thinner (e.g., hypertonic saline or dornase alfa (PULMOZYME®)), a CFTR modulator (e.g., ivacaftor (KALYDECO®), lumacaftor, lumacaftor/ivacaftor (ORKAMBI®), tezacaftor/ivacaftor (SYMDEKO®), or TRIKAFTA® (elexacaftor/ivacaftor/tezacaftor)), a mucolytic (e.g., acetylcysteine, ambroxol, bromhexine, carbocisteine, erdosteine, mecysteine, and dornase alfa), an immunosuppressive agent, normal saline, hypertonic saline, or a combination thereof.

For example, any one the compositions described herein may be administered in combination with one or more immunosuppressive agents. Any suitable immunosuppressive agent may be used. For example, non-limiting examples of immunosuppressive agents include corticosteroids (e.g., an inhaled corticosteroid (e.g., beclomethasone (QVAR®), budesonide (PULMICORT®), budesonide/formoterol (SYMBICORT®), ciclesonide (ALVESCO®), fluticasone (FLOVENT HFA®), fluticasone propionate (FLOVENT DISKUS®), fluticasone furoate (ARNUITY ELLIPTA®), fluticasone propionate/salmeterol (ADVAIR®), fluticasone furoate/umeclidinium/vilanterol (TRELEGY ELLIPTA®), mometasone furoate (ASMANEX®), or mometasone/formoterol (DULERA®), prednisone, or methylprednisone), polyclonal anti-lymphocyte antibodies (e.g., anti-lymphocyte globulin (ALG) and anti-thymocyte globulin (ATG) antibodies, which may be, for example, horse- or rabbit-derived), monoclonal anti-lymphocyte antibodies (e.g., anti-CD3 antibodies (e.g., murmomab and alemtuzumab) or anti-CD20 antibodies (e.g., rituximab)), interleukin-2 (IL-2) receptor antagonists (e.g., daclizumab and basiliximab), calcineurin inhibitors (e.g., cyclosporin A and tacrolimus), cell cycle inhibitors (e.g., azathioprine, mycophenolate mofetil (MMF), and mycophenolic acid (MPA)), mammalian target of rapamycin (mTOR) inhibitors (e.g., sirolimus (rapamycin) and everolimus), methotrexate, cyclophosphamide, an anthracycline (e.g., doxorubicin, idarubicin, aclarubicin, daunorubicin, epirubicin, valrubicin, mitoxantrone, or a combination thereof), a taxane (e.g., TAXOL® (paclitaxel)), and a combination thereof (e.g., a combination of a calcineurin inhibitor, a cell cycle inhibitor, and a corticosteroid).

The compositions described herein may be administered to a mammal alone or in combination with pharmaceutically acceptable carriers. As noted above, the relative proportions of active ingredient and carrier are determined by the solubility and chemical nature of the compound, chosen route of administration and standard pharmaceutical practice.

The dosage of the present compositions will vary with the form of administration, the particular compound chosen and the physiological characteristics of the particular patient under treatment. It is desirable that the lowest effective concentration of virus be utilized in order to reduce the risk of undesirable effects, such as toxicity.

Augmenters: The compositions can be used in combination with augmenters of AAV transduction to achieve significant increases in transduction and/or expression of nucleic acid molecules (transgenes). Any suitable augmenter can be used. For example, U.S. Pat. No. 7,749,491, which is incorporated by reference herein in its entirety, describes suitable augmenters. The augmenter may be a proteasome modulating agent. The proteasome modulating agent may be an anthracycline (e.g., doxorubicin, idarubicin, aclarubicin, daunorubicin, epirubicin, valrubicin, or mitoxantrone), a proteasome inhibitor (e.g., bortezomib, carfilzomib, and ixazomib), a tripeptidyl aldehyde (e.g., N-acetyl-1-leucyl-1-leucyl-1-norleucine (LLnL)), or a combination thereof. In some embodiments, the augmenter is doxorubicin. In other embodiments, the augmenter is idarubicin.

The compositions, e.g., EVAAV and the augmenter(s) may be contacted with a cell, or administered to a subject, in the same composition or in different compositions (e.g., pharmaceutical compositions). The contacting or the administration of the compositions and the augmenter(s) may be sequential or simultaneous.

### EXAMPLES

### Example 1: Extracellular Vesicle-Associated, Genetically Engineered Adeno-Associated Virus 6 as a Gene Delivery Platform for Inhaled Gene Therapy

Extracellular vesicles (EVs) are naturally occurring molecular vesicles produced and released from plant and animal cells and play important roles in intercellular communication (1). Due to its ability to shuttle various cargoes, including metabolites, proteins, and nucleic acids, through extracellular milieus and between cells, EV has been widely investigated as a vehicle for enhancing therapeutic delivery (2, 3).

The inventors have recently discovered that EVs are capable of efficiently penetrating unperturbed airway mucus samples collected from patients with muco-obstructive lung diseases (MOLDs), leading to the hypothesis that EV would enhance the inhaled gene transfer efficacy of AAV if the AAV externally associated with the EV does not compromise the mucus-penetrating property of EVs.

To test this hypothesis, EVAAVs were prepared based on AAV serotype 6 (AAV6) leveraging on the inventors previous finding that AAV6, unlike other widely used AAV serotypes, possesses unique ability to resist mucoadhesion. The diffusion rates of EV and EVAA6 in human airway mucus were confirmed to be virtually identical, underscoring that AAV6 association did not negatively impact on the newly discovered ability of EV to efficiently traverse the airway mucus gel. Encouraged by this observation, a pilot study was conducted to investigate whether piggybacking on EV would enhance the transduction efficiency of AAV6 in mucus-covered airway epithelial cells in vitro and in vivo. In this study, it was found that EV-associated AAV6 (EVAAV6) provided significantly greater reporter transgene expression in mucus-covered air-liquid interface (ALI) cultures of primary human airway epithelial (HAE) cells and in mouse lungs (following intratracheal administration) compared to standard (i.e., EV-free) AAV6. It was noted that pre-existing neutralizing antibody (Nab) not only compromises cellular uptake but also can promote AAV entrapment in airway mucus via multivalent interactions between N-glycan-rich Fc regions of virus-associated NAb and mucin fibers (11-13). However, it is expected that such effects would be at least partially evaded by the association of AAV with EVs, in conjunction with capsid engineering, which will be tested for use of this hybrid system for patients with pre-existing AAV-specific immunity or for repeated administration. Embracing the limited packaging capacity of AAV (i.e., 4.7 kb), we will also assess the ability of EVAAV6 and newly engineered mutant AAV6 vectors (mAAV6) piggybacked on EVs (EVmAAV6) to deliver two reporter cassettes individually packaged in individual AAV vectors into the same cells for potential delivery of large genes beyond the natural AAV packaging capacity (e.g., CFTR gene).

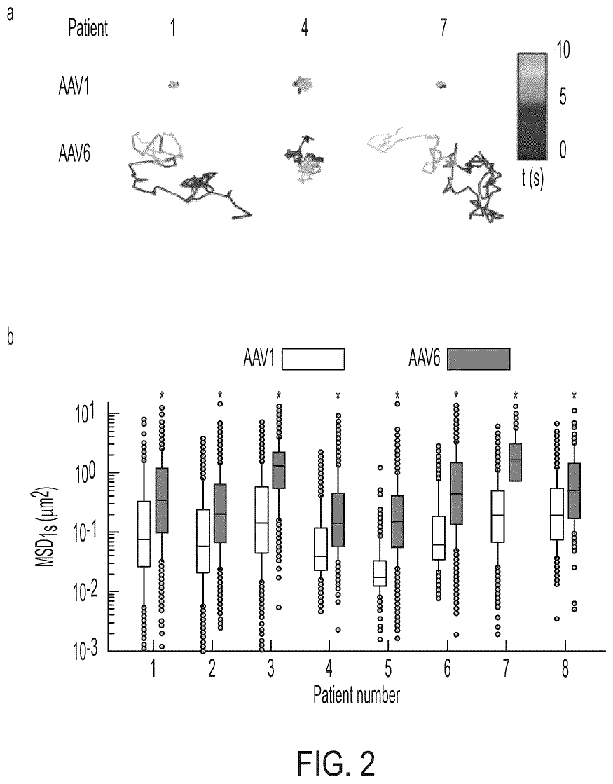

Non-mucoadhesive AAV6 as a background serotype for engineering novel EVmAAV6. AAV is a non-pathogenic parvovirus that has been most widely investigated as a virus-based delivery platform for safe, effective and long-lasting gene therapy of numerous diseases affecting different organs. Promising outcomes in preclinical studies have led to numerous human clinical trials (16-19), and FDA has recently approved AAV-based gene therapy medicines for the treatment of a rare eye disease and spinal muscular atrophy (20-22). However, such clinical breakthrough is yet to be realized for inhaled gene therapy of chronic MOLDs, such as asthma, cystic fibrosis (CF) and chronic obstructive pulmonary disease (COPD), due to inability of clinically tested gene vectors to provide therapeutically relevant gene transfer efficacy in the lung (23-26). The only AAV serotype tested in clinical trials of inhaled gene therapy to date, AAV2, is not efficient in transducing airway cells in human lungs and readily rejected by Nab upon initial and/or subsequent treatment(s) (23, 27). While underappreciated in early clinical studies, airway mucus is now recognized as a rate-limiting extracellular barrier to inhaled therapeutics (28-38). Airway mucus is essentially a “sticky net”, primarily composed of a dense mesh of mucin fibers containing negatively charged glycosylated domains flanked by periodic hydrophobic regions (39), which forms an adhesive and steric barrier to inhaled foreign matters. The barrier properties are further reinforced in the lungs of patients with MOLDs characterized by chronic inflammation and mucus accumulation (23, 29, 37, 40, 41). Inhaled gene vectors trapped in the airway mucus cannot efficiently traverse and distribute throughout the lung airways and are rapidly removed from the lung by physiological mucus clearance mechanisms prior to reaching the underlying airway cells (29, 37). Indeed, we have demonstrated that many of leading and/or clinically tested gene vectors, including various AAV serotypes (i.e., AAV1, 2 and 5 (12, 42)), adenovirus (42), and polymeric non-viral gene vectors (43, 44), cannot efficiently penetrate human airway mucus collected from patients with MOLDs (i.e., sputum). Of note, sputum samples serve as a reliably representative ex vivo model of pathologically thickened human airway mucus barrier, which is a common feature of lung airways of MOLDs. Collectively, the airway mucus is now universally appreciated as one of the key obstacles that must be overcome by inhaled gene vectors to achieve clinically relevant inhaled gene delivery efficacy (23, 29, 37, 38). Encouragingly, the inventors have discovered that AAV6, unlike other serotypes, possesses unique ability to avoid mucoadhesion and thus efficiently percolate the human airway mucus (FIGS. 2A, 2B). It was found that AAV6 exhibited significantly and uniformly enhanced diffusion rates in sputum samples collected from multiple independent patients compared to AAV1, a serotype shown to outperform the clinically tested AAV2 (45, 46) (10). As a result, AAV6 provided markedly more widespread and greater reporter transgene expression in mucus-covered ALI cultures of primary HAE cells and in mucus-plugged lung airways of transgenic mouse model of MOLDs, compared to AAV1 (10). Of note, AAV6 binds to α-2,6-linked sialic acid (47) which is rich on the apical surface of human airway epithelial cells (48). To this end, AAV6 constitutes an excellent background serotype to implement EV piggybacking and capsid engineering for enhanced inhaled gene transfer efficacy, supposed that the non-mucoadhesive capsid property is retained.

Recombinant library of capsid optimized AAV rationally designed to promote intracellular trafficking to cell nuclei. Proteasome degradation of AAV capsid protein is a primary intracellular hurdle that compromises transduction efficiency of AAV-based vectors following endocytic uptake regardless of the target cell types (49-54). Of note, intact AAV capsid is required for efficient delivery of DNA payloads to cell nuclei, particularly more so for post-mitotic cells, such as various lung airway cells (55). To address this critical restriction, proteasome inhibitor or capsid modification has been employed and shown to enhance the ability to AAV vectors to mediate transgene expression and/or to avoid AAV-specific immune responses by reducing antigen presentation (54, 56-60). 29 conservative amino acid positions on the AAV capsid were identified that tolerate mutagenesis (FIGS. 2A, 2B). Several mutations were introduced to different AAV serotypes, and the improvement in vector performances was validated. Specifically, these mutant vectors were demonstrated to facilitate intracellular processing and trafficking to nuclei (62, 65, 68, 69) and/or reduce AAV-specific immune responses (70, 71) by minimizing the proteasome degradation of AAV vectors. It was thus proposed to engineer a panel of self-encoding mutants, backgrounded on the non-mucoadhesive AAV6, to possess one or more (up to three) substitution(s) at each of 29 conserved phosphorylation sites with 20 naturally-occurring amino acids. Mutations in the panel are located on the AAV capsid surface to potentially contribute to vector interactions with surrounding biological entities, including Nab, cells and mucus gel. mAAV6 will be screened for their ability to resist mucoadhesion while enhancing the nuclear delivery of DNA payloads, using complementary models, including ALI culture of primary HAE cells, sputum samples collected from patients with MOLDs and the above-mentioned mucus-plugged mouse model (72). It is expected that new mAAV6 candidates would be identified that provide enhanced intracellular trafficking, reduced immunogenicity and non-mucoadhesive surface, which will be used to prepare EVmAAV6 for further in vivo evaluations. Overall, it is hypothesized that combination of (i) piggybacking of non-mucoadhesive AAV6 on EVs and (ii) rational capsid_engineering to facilitate intracellular trafficking will collectively result in an innovative hybrid delivery platform capable of overcoming multiple biological delivery barriers to provide unprecedented, inhaled gene transfer efficacy

TABLE 1
Established or potential benefits
of individual components of EVmAAV6

| Individual | |
|---|---|
| components | Benefits |
| EV | Mucus penetration |
| | Enhanced cellular uptake |
| | Resistance to interactions with vector-specific |
| | NAb |
| | Potential to deliver a large (>4.7 kb) DNA |
| | payload by association of dual vector systems |
| | with the same EVs |
| | Potential to reduce the clinical dose |
| AAV6 capsid | Mucus penetration |
| | Natural affinity to the apical surface of human |
| | airway epithelial cells |
| Capsid optimization | Enhanced nuclear delivery of the encaged |
| | DNA payload |
| | Reduced vector-specific immune responses |
| | (by preventing antigen presentation) |

Proof-of-principle preclinical therapeutic efficacy study: thymulin gene therapy for treating allergic asthma. Thymulin provides remarkable anti-inflammatory and anti-fibrotic effects in the lungs of a preclinical model of allergic asthma. Specifically, the inventors found that thymulin-expressing plasmids delivered by the newly engineered polymeric gene vectors (73) reversed key pathological hallmarks of allergic asthma, including asthmatic inflammation, pulmonary fibrosis, and mechanical dysregulation, in the lungs of an ovalbumin (OVA)-based allergic asthma model, primarily by immunomodulation (74)). Thus, it is proposed to investigate the therapeutic efficacy of EVAAV6 and lead EVmAAV6 carrying a transgene expression cassette encoding the thymulin to establish proof-of-principle clinical relevance. Despite being widely used in preclinical settings, it is noted that OVA-based models hold inherent limitations, including weak relevance to human asthma-causing allergens, lack of continuous challenge and relatively short-lived nature (75). To this end, therapeutic approach will be evaluated additionally in a more human asthma-like preclinical model, specifically mouse model of chronic allergic asthma established by continuous inhaled challenges with house dust mite (HDM) (76-78). While the primary goal of is to develop a hybrid gene delivery platform universally applicable to MOLDs, successful outcomes in this efficacy study will pave a way for clinical development of inhaled gene therapy of highly refractory chronic asthma.

Development of EVmAAV6 as a novel gene delivery platform for inhaled gene therapy of MOLDs. As summarized in Table 1, association with EVs potentially enhances AAV6 transduction in mucus-covered lung airways in a multi-pronged manner. EVAAV6 is capable of efficiently penetrating the airway mucus regardless of whether AAV6 is associated with EVs internally or externally, due to the mucus-penetrating properties of both components. Likewise, EVAAV6 can synergistically promote uptake of the DNA payload by lung airway cells, following inhaled administration, due to the natural ability of EVs to be taken up by various cells as well as the inherent affinity of AAV6 to the apical receptors on lung airway cells. Of note, EVAAV, irrespective of serotype, has not been investigated for inhaled gene therapy applications until our recent pilot study demonstrating that EVAAV6 provides markedly greater reporter transgene expression in mouse lungs compared to AAV6 following intratracheal administration. EVAAV6 may also bypass neutralization by pre-existing AAV-specific NAb, increase packaging capacity, reduce clinical doses and/or enable repeated dosing to mediate sustained transgene expression. To this end, we expect our proposal to yield a novel and innovative hybrid gene delivery platform that provides unprecedented gene transfer efficacy potentially with broad applicability.

Investigation of a recombinant panel of rationally-designed, capsid-optimized AAV6 variants. Prior studies of novel AAV vector development involve screening of libraries composed of randomly introduced mutations. Here, a recombinant library of rationally designed, self-encoding mAAV6 (i.e., ~3×10⁷ different variants) is established. The mutants possess one or up to three single amino acid substitution(s) at 29 phosphorylation sites where mutation in each location has been confirmed not to compromise viral particle structure/integrity and general infectivity, while endowing AAV vectors with an additional functional arm to facilitate intracellular trafficking to cell nuclei, ultimately to enhance gene transfer efficacy.

Use of most relevant in vitro, ex vivo and in vivo experimental settings to develop clinically-relevant inhaled gene delivery strategies. Limited preclinical-to-clinical correlation observed in many of the inhaled gene delivery studies is largely attributed to the lack of a single experimental model reliably recapitulating delivery hurdles found in relevant human lung disease conditions. Here, it is proposed to use complementary in vitro (mucus-covered ALI cultures established with airway cells harvested from patients), ex vivo (unperturbed sputum freshly collected from patients) and in vivo (mouse model exhibiting chronic inflammation and airway mucus hypersecretion) models concurrently to evaluate and screen mAAV6 and EVmAAV6 in experimental settings exhibiting key delivery and immunological barriers found in the lungs of patients with MOLDs.

Potential development of novel immunomodulatory gene therapy for treating asthma. The proof-of-principle study shows that inhaled thymulin gene therapy, due to its unique immunomodulatory properties, provides compelling therapeutic benefits in a mouse model of OVA-induced allergic asthma. We expect to establish promising therapeutic efficacy in a more human asthma-like model (i.e., HDM-challenged chronic allergic asthma model) via efficient thymulin transgene expression mediated by our lead EVmAAV6. Despite not being the primary goal of this proposal, successful execution of this study may promote clinical development of long-lasting gene therapy of asthma.

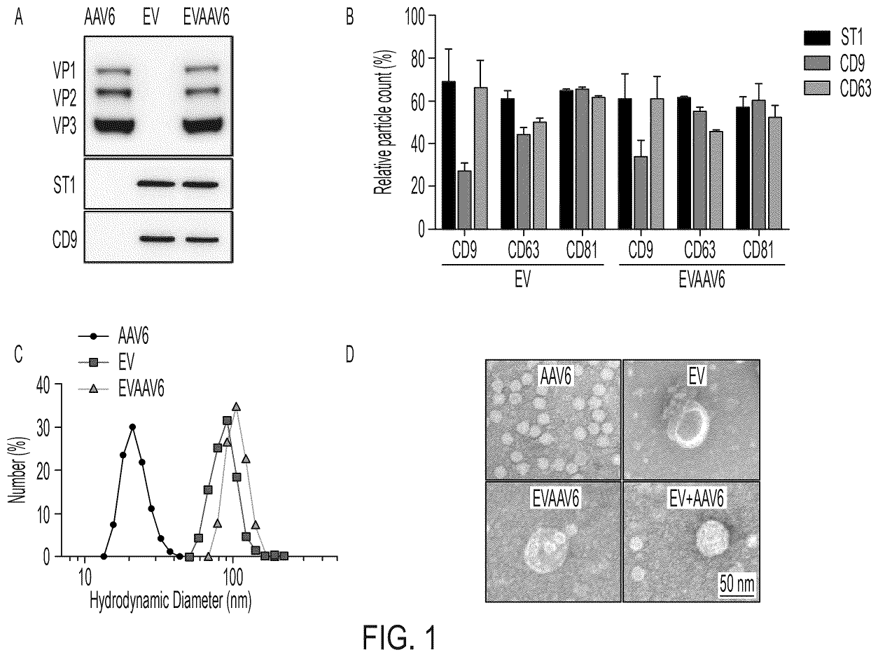

EVAAV6 efficiently penetrates human airway mucus and provides markedly greater transgene expression compared to AAV6 in a human bronchial epithelial cell line. Based on the pilot observation that EVs are capable of efficiently penetrating human airway mucus, it was hypothesized that EVAAV prepared with AAV vectors with non-mucoadhesive capsids (e.g., AAV6; FIGS. 2A, 2B) would efficiently penetrate the human airway mucus as well. EVAAV6, was first prepared as described herein, and comprehensive characterization was conducted using western blot analysis, tetraspanin chips, Spectradyne particle analyzer and dynamic light scattering (DLS). It was confirmed that EV and EVAAV6 similarly expressed syntenin-1 (ST-1) and CD9 (FIG. 1A), providing evidence that both are similarly exosome-rich EV populations.

Transmission electron microscopy (TEM) revealed that AAV6 was internally and externally associated with EV for EVAAV6 produced in HEK293 cells, whereas post-mixture of individually prepared EV and AAV6 (EV+AAV6) showed no association. In parallel, DLS analysis showed greater hydrodynamic diameters of EVAAV6 compared to EV, reflecting the presence of the externally associated AAV6 on EVAAV6. For this hypothesis, EV and EVAAV6 were fluorescently labeled, and their diffusion behaviors were investigated in CF sputum using multiple particle tracking (MPT). It was found that both EV and EVAAV6 exhibited the mean square displacement (MSD) values greater than those of mucus-penetrating AAV6 on average, underscoring that EV further enhanced the mucus-penetrating ability of AAV6 upon their association. Transduction efficiency of AAV6, EVAAV6 and EV+AAV6 was next compared in human lung airway cell line (i.e., human bronchial epithelia or HBE cells). At all multiplicity of infection (MOI) tested, EVAAV6, but not EV+AAV6, provided roughly two orders of magnitude greater luciferase transgene expression in HBE cells compared to AAV6.

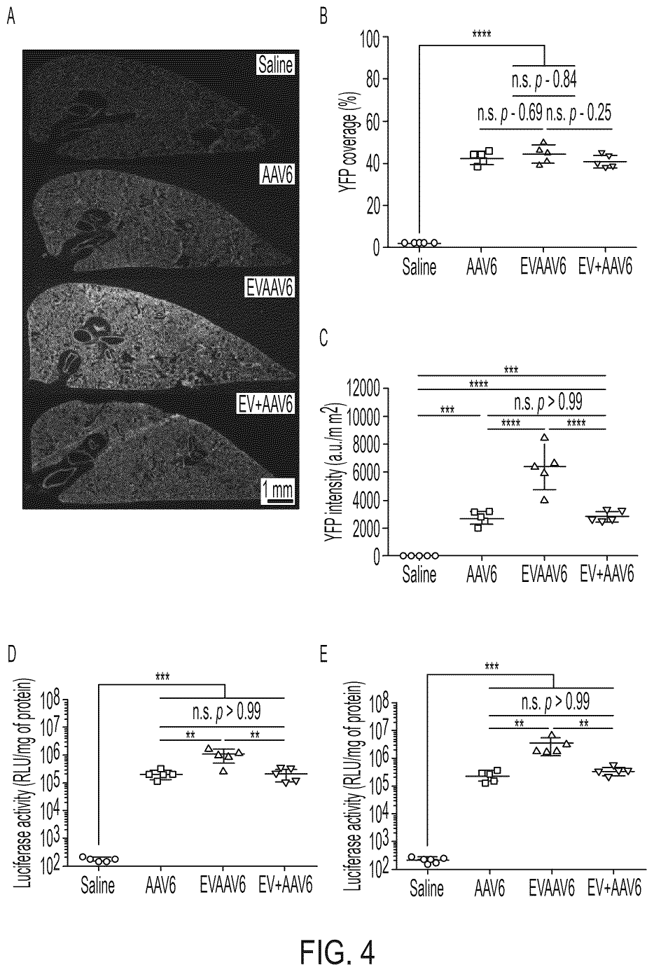

EVAAV6, but not EV+AAV6, provides significantly greater transgene expression compared to AAV6 in mouse lungs following intratracheal administration. It was then investigated whether the enhancements in mucus penetration and cellular transduction enabled by EV piggybacking (FIGS. 4A-4E) resulted in the improvement of reporter transgene expression in the lung in vivo. Briefly, C57BL/6 mice intratracheally received AAV6, EVAAV6 or EV+AAV6 at a dose of 4×10⁹ GC per animal and lung tissues were harvested 2 weeks after the administration for the assessment of reporter transgene expression. Lung tissues from individual animals were then cryosection and imaged under a confocal microscope without amplification of the signal with immunohistochemical staining. The representative tile-scanned images showed that all three treatments mediated uniform YPF transgene expression, but EVAAV6 exhibited greater fluorescence intensity compared to the other groups (FIG. 4A), which was quantitatively validated by Image-based quantitative analysis with a custom-written MATLAB software (10) (FIG. 4B). In a separate set of animals, it was also found that EVAAV6 provided an order of magnitude greater luciferase activity compared to AAV6 and EV+AAV6 (FIG. 4C).

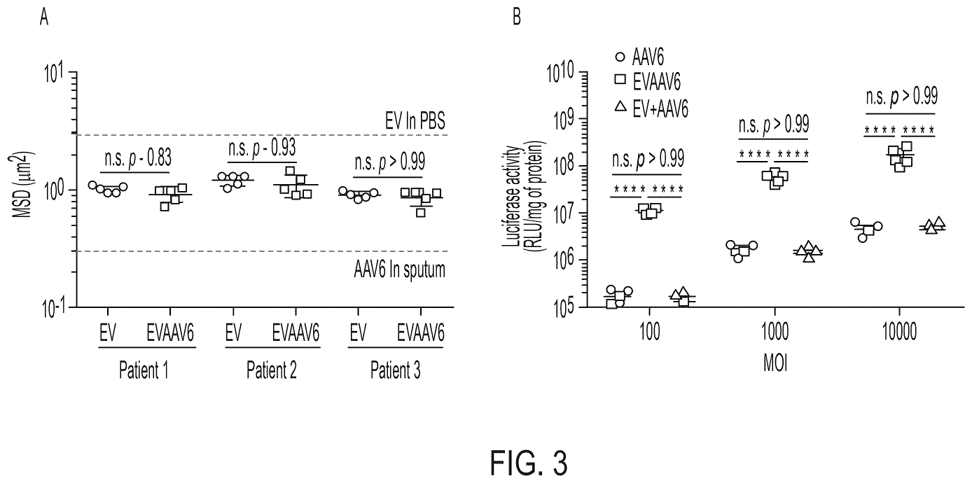

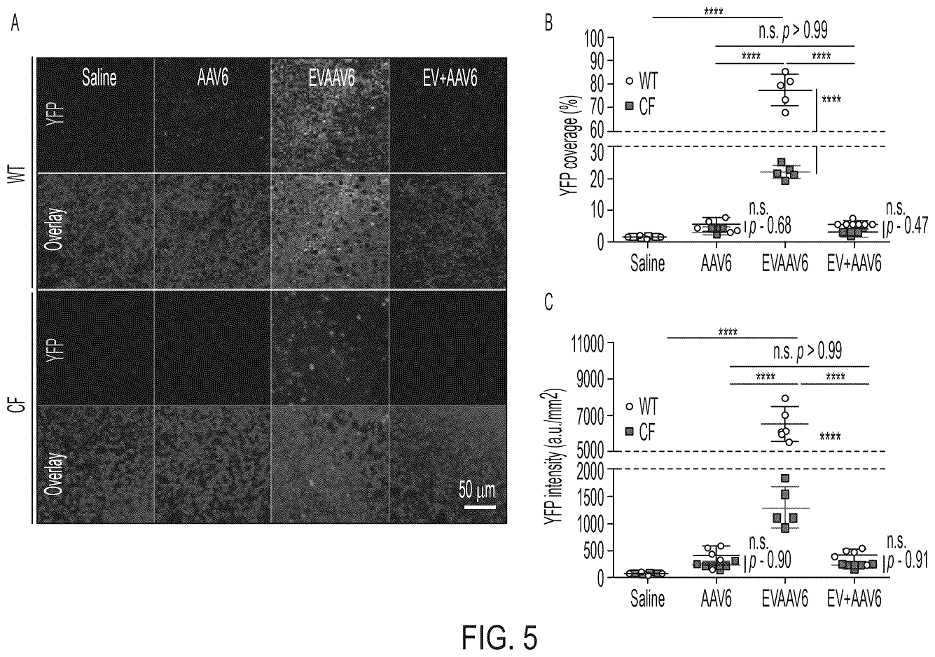

EVAAV6, but not EV+AAV6, provides significantly greater coverage and level of transgene expression in mucus-covered ALI cultures of primary HAE cells compared to AAV6. For clinical relevance, it is critical that our hybrid delivery strategy enhances transduction of lung airway cells found in human lungs. To test this, we assessed the ability of AAV6, EVAAV6 and EV+AAV6 to transduce mucus-covered ALI culture of primary HAE cells, specifically bronchial epithelial cells. Of note, the model uniquely emulates the physiological human lung airways and associated biological barriers, thereby serving as an excellent testbed for evaluating the performance of lung-directed gene delivery platforms (10, 23, 9). Representative confocal images revealed that EVAAV6, but not EV+AAV6, mediated more uniform and enhanced reporter transgene expression compared to AAV6, in agreement with the in vivo observations. Quantitatively, EVAAV6 exhibited markedly and significantly greater coverage and level (i.e., intensity) of transgene expression compared to AAV6 and EV+AAV6. The series of the in vitro and in vivo analyses (FIGS. 3A, 3B, 4A-4E, 5A-5C) consistently and collectively suggests that EV markedly enhances the ability of AAV6 to transduce lung airway cells by breaching multiple biological delivery barriers. Further, it appears that the association of EV and AAV6 during the production, rather than post-mixture after independent preparation of EV and AAV6, is essential for the enhancement.

Capsid optimization significantly improves in vitro gene transfer efficacy of AAV6 by enhancing intracellular trafficking to cell nuclei. In parallel to the EV piggybacking strategy, capsid-optimized mAAV6 was engineered to facilitate the intracellular trafficking of DNA payloads loaded in the capsid to cell nuclei as an additional means to enhance transduction efficiency. Specifically, at least three different mAAV6 were constructed carrying one or two mutation(s), including AAV6-T492V, AAV6-S663V and AAV6-492V+S663V, that exhibited significantly greater in vitro transduction efficiency compared to wild-type AAV6. Of note, the AAV6-T492V+S663V harbors two mutations that camouflage the AAV6 capsids from phosphorylation by intracellular kinases, thereby reducing proteasome degradation. A marked increase was confirmed in the nuclear fraction of the mutant compared to wild-type AAV6, suggesting that its unique ability to resist the proteasome degradation facilitated intracellular trafficking of the vector to cell nuclei. In addition, the proteasome degradation promotes AAV capsid presentation by antigen-presenting cells and thus the vector-associated immune responses. To this end, the ability to minimize the intracellular degradation is an additional mechanism by which AAV-mediated gene transfer efficacy is enhanced while potentially reducing vector-specific immune responses (71). It was also confirmed that another mutant, AAV6-S663V, provided marked greater reporter transgene expression in primary human airway cells, including basal cells, compared to wild-type AAV6, presumably by enhancing nuclear import of the vector. It is thus expected that EV piggybacking and capsid engineering will synergistically enhance lung airway transduction.

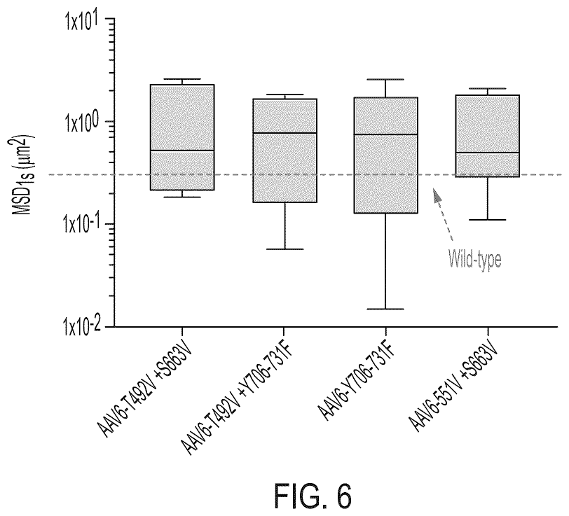

Multiple capsid-optimized mAAV6 efficiently penetrate human airway mucus. Given that newly engineered mAAV6 would be both internally and externally associated with EV to form EVmAAV6, it is critical that mAAV6 retains the unique non-mucoadhesive property inherent to wild-type AAV6 (FIGS. 2A, 2B and (10)). Using MPT the diffusion rates of several mAAV6 were thus quantitatively evaluated, including one that confirmed for significantly improved intracellular trafficking and cellular transduction (AAV6-T492V+S663V, in multiple independent CF sputum samples. It was found that all these mAAV6 exhibited similar or greater MSD values on average compared to wild-type AAV6 (FIG. 6), suggesting that the capsid optimization did not compromise the non-mucoadhesive property of the parent wild-type AAV6.

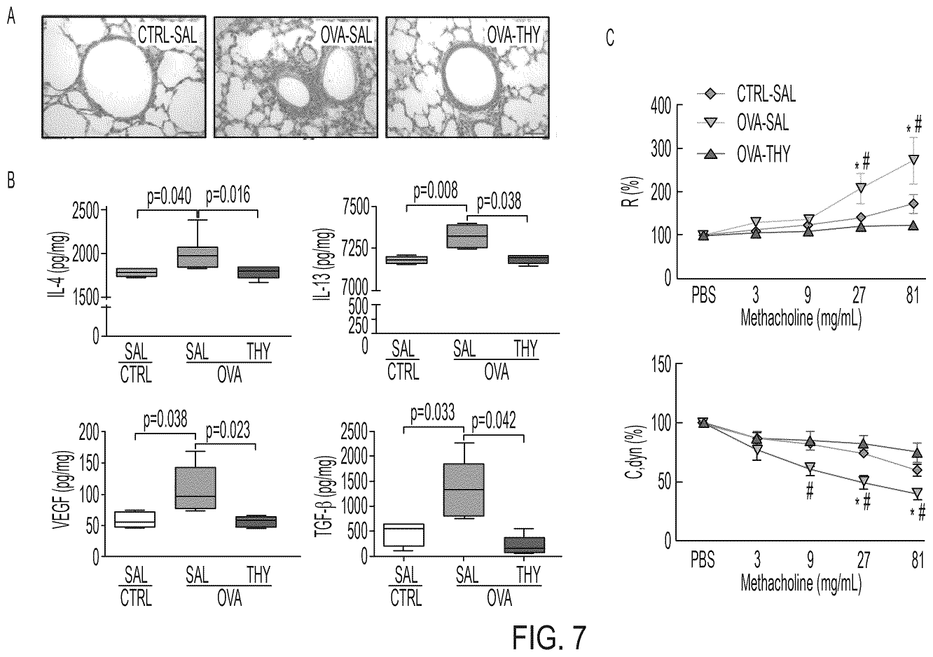

Inhaled thymulin gene therapy normalizes key pathological features in the lungs of a mouse model of allergic asthma. The inventors have recently demonstrated that inhaled thymulin gene therapy resolves key pathological outcomes, including allergic (i.e., T helper type 2 or Th2) inflammation, pulmonary fibrosis and mechanical dysregulation, manifested in a mouse model of OVA-induced allergic asthma in a therapeutic manner (74). The model was established with sequential sensitization and challenge with OVA (81) and it was confirmed that relevant disease phenotypes were all fully established and retained during the study period. Animals were then intratracheally treated with the synthetic nanoparticles developed to efficiently penetrate human airway mucus (73) carrying thymulin-expressing plasmids. It was first histologically determined that immune cell infiltration, mucus hypersecretion and airway constriction were normalized by the therapy (FIG. 7A). In parallel, bronchoalveolar fluid (BALF) was harvested from mice from each group and analyzed the levels of Th2 cytokines, including IL-4 and IL-13, and pro-fibrotic mediators, including VEGF and TGF-β, using enzyme-linked immunosorbent assay (ELISA). The levels of all these key soluble factors were significantly elevated in the model (OVA-SAL group), as observed in the lungs of patients with allergic asthma, but the inhaled thymulin gene therapy (OVA-THY group) brought them all down to the levels observed with healthy mice (CTRL-SAL group) (FIG. 7B). It was also found that the levels of chemokines for both eosinophils (CCL11 or eotaxin) and neutrophils (CXCL1) elevated in the lungs of asthmatic mice were normalized by the thymulin gene therapy (74).

The methacholine challenge test was conducted next, commonly performed in the clinic to evaluate the lung function of patients with asthma (82). The airway hyperresponsiveness (AHR) in the asthmatic lungs undergoing fibrotic process (83) was confirmed by more pronounced dose-dependent increase in airway resistance and decrease in dynamic compliance (C, dyn). It was found that the thymulin gene therapy normalized the AHR, underscoring that the mechanical dysregulation was resolved in this model (FIG. 7C). Finally, it was discovered that the compelling and comprehensive therapeutic benefits were achieved primarily by immunomodulatory effects of the thymulin gene therapy. Specifically, the therapy mediated therapeutically favorable phenotypic shifts of key immune cells: lymphocytes from pathological Th2 to therapeutic regulatory T (Treg) subtype and macrophages from M2 to M1 subtype (74). To this end, thymulin constitutes an excellent therapeutic agent for establishing proof-of-principle clinical relevance of the lead EVmAAV6 to be developed in this study.

### Example 2: Research Strategy

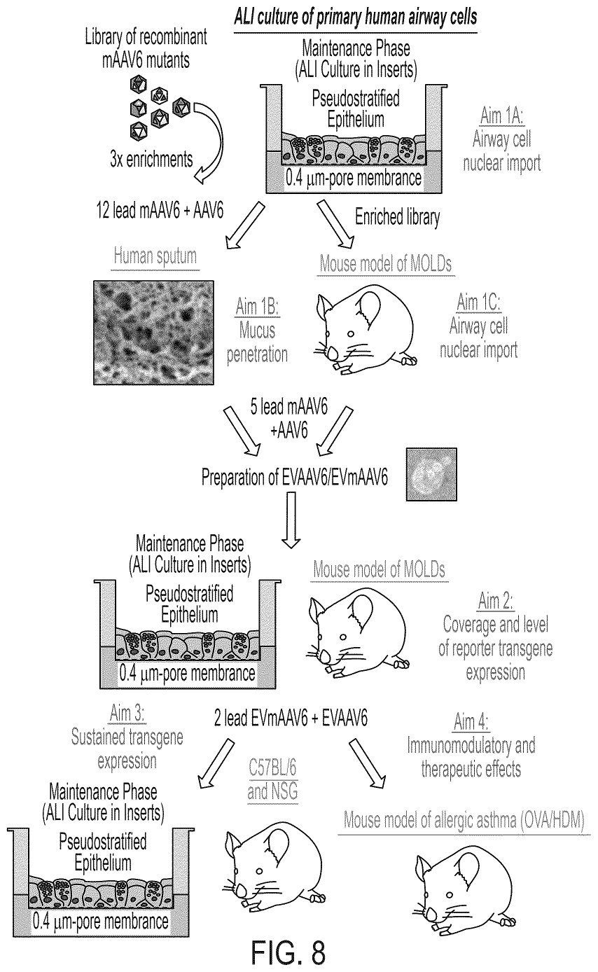

The overall goal of this study is to develop and evaluate novel hybrid gene delivery platform based on EVmAAV6 for inhaled gene therapy of MOLDs (FIG. 8). A recombinant library of capsid-optimized mAAV6 be constructed providing enhanced intracellular trafficking to cell nuclei, while retaining the non-mucoadhesive capsid property of wild-type AAV6. The mutants will then be extensively screened using complementary experimental models closely emulating MOLDs (e.g., CF, COPD, and asthma) to determine lead candidates providing efficient delivery of DNA payloads to cell nuclei of mucus-covered lung airway cells. Those testbeds include mucus-covered ALI cultures of primary HAE cells (in vitro), human airway mucus freshly collected from patients (ex vivo) and a mucus-plugged mouse model of MOLDs (in vivo). First, the mAAV6 candidates will be screened using the ALI culture, and a few selected mutants will be evaluated for efficient penetration through unperturbed sputum samples spontaneously expectorated by patients with CF and possibly patients with COPD or asthma if available. A mutant library will be screened the animal model to determine lead mAAV6. Subsequently, EVmAAV6 will be prepared based on lead mAAV6 candidates, which will be assessed for coverage and overall level of reporter transgene expression using ALI cultures of primary HAE cells and the mucus-plugged mouse model of MOLDs. Two winners achieving either greater coverage or overall level will then be investigated for the ability to deliver two reporter cassettes individually packaged in mAAV6 into the same cells in vitro and in vivo for potential delivery of a large gene beyond the natural packaging capacity of AAV. In parallel, the lead EVmAAV6 will be evaluated for their ability to provide long-term transgene expression by transducing airway progenitor cells or by allowing repeated treatment without being inactivated by the host immune system. These lead EVmAAV6 will be constructed to carry thymulin-encoding DNA cassette and evaluated for therapeutic efficacy in well-established mouse models of allergic asthma. Number of animals to be used for each proposed study is determined by power calculations based on our preliminary data (5% significance level and 90% power). Studies will be performed with both male and female animals in equal proportions. Two-tailed Student's t-tests (assuming an unequal variance) and ANOVA, followed by post-hoc analysis, will be executed for two- and multi-group comparisons, respectively.

Screen a panel of newly engineered recombinant mAAV6 for mucus penetration and nuclear import in vitro, ex vivo and/or in vivo. Engineer and screen capsid-optimized, self-encoding mAAV6 for (A) nuclear import in mucus-covered ALI cultures of primary human airway cells in vitro, (B) penetration through human airway mucus ex vivo and (C) nuclear import in lung airway cells of a transgenic mouse model exhibiting an enhanced mucus barrier manifested in human MOLDs.

A panel of capsid-optimized mAAV6 that potentially enhance intracellular trafficking to cell nuclei, while retaining the capsid integrity will be established. Specifically, a library of mAAV6 will be generated by substituting one or up to three amino acid(s) at 29 conserved positions.

The previously implemented substitutions were based on similarity in the amino acid structure, but here these will replace amino acids at these positions with all 20 naturally occurring amino acids used in protein synthesis in humans. The theoretical complexity of all these permutations would be approximately 3×10⁷, which is relatively lower than the previously reported capsid recombinant libraries (84-92). However, most of them will likely possess intact capsid structure and inherent infectivity of wild-type AAV6 due to the implementation at conserved sites only, providing a sufficient number of variations to be screened. Of note, the library will be prepared by site-directed mutagenesis on already constructed plasmids with randomized primers and subsequent self-packaging of all mutants simultaneously in AAV-producing HEK293 cells at a low capsid-mutated plasmid library to helper plasmid ratio of 1:5,000 to ensure self-packaging while minimizing the probability of possible cross-packaging (93).

Upon the preparation of the library, recombinant mAAV6 will be screened first for intracellular trafficking to cell nuclei using mucus-covered ALI cultures of human primary airway cells. Human primary airway cells will be obtained from the CF Clinical and Translational Center at University of Alabama at Birmingham (Dr. Steven Rowe). Cells will be plated apically on collagen coated 6-well semi-permeable transmembrane snapwell culture inserts and grown submerged in media for 3-4 days until the cells reach ~100% confluency. The apical media will then be removed to create an ALI culture where cells will be differentiated to mimic a polarized conducting epithelium found in human lung airways (10). The cells will form a monolayer with high transepithelial electrical resistance (TEER; 500-700 Ω·cm²), an indicative of intact tight junctions (94). The ALI cultures will be prepared with CF (and potentially COPD) airway cells, but it would be challenging to obtain airway cells from asthma patients generally not undergoing lung transplant. However, the model should serve as a reliable representative model to test the ability of mAAV6 to resist adhesion to pathologically-thicken airway mucus gel and deliver self-encoding DNA payloads into the nuclei of human primary airway cells. To obtain NAb against wild-type AAV6, 1×10¹⁰-10¹¹ wildtype AAV6 will be intravenously injected into C57BL/6 mice, serum harvested 2-3 weeks after the injection and determine the NAb titer using ELISA (95). The library of mAAV6 will be added to the apical surface of the ALI culture, with or without pre-incubation with serum containing anti-AAV6 NAb, and cells will be harvested two days after the administration (i.e., sufficient time for cell uptake and nuclear localization if any). The nuclear fraction will be isolated and genomic DNA extracted, followed by PCR amplification with AAV6 capsid specific primers and evaluation of relevant abundance of associated mutants by sequencing of individual clones. Of note, mAAV6 in the nuclear fraction are those successfully penetrate the apical mucus gel layer, internalize into airway cells, bypass proteasome degradation and traffic to cell nuclei. Three rounds of enrichment procedure will be conducted where capsid specific primers will be used to amplify and re-package only variants isolated from previous selection steps. The selection criteria will be applied to collect clones with over 5%, 10% and 25% frequency at round one, two and three, respectively, to determine up to 12 lead mAAV6 candidates providing greatest nuclear import in primary HAE cells. The selection process is straightforward, as it involves infection of the ALI culture simultaneously with the whole library, followed by selection and analysis of lead mutants via high-throughput next-generation sequencing.

An additional selection pressure will be applied to ensure that the mAAV6 selected resist mucoadhesion and thus not to compromise the mucus-penetrating property of EV upon external association. Briefly, the diffusion rates of up to 12 mutants selected will be quantified in unperturbed sputum samples spontaneously expectorated by patients vising the Johns Hopkins Adult CF clinic using MPT, which have been routinely conducted (10, 44, 73, 96, 97). Alexa Fluor 647-labeled AAV vectors will be added to freshly collected sputum samples and x- and y-coordinates of particle trajectories will be recorded over time to calculate MSD values. Of note, the labeling strategy does not alter the surface property and infectivity of AAV (12). Sputum samples will be stored at 4° C. for <24 hours after the collection to ensure that the biophysical properties are retained (29). The MSD value previously determined for wild-type AAV6 (i.e., MSD >0.3 μm² at a timescale of 1 second; (10)) will be considered “non-mucoadhesive” and thus serve as a minimal inclusion criterion.

The lead mAAV6 desired for human lung-directed gene therapy that also provides favorable performances in a mouse model of MOLDs will be selected. To do this, the library enriched over three rounds in primary HAE cell nuclei will be used (i.e., top 10%) to determine mAAV6 candidates providing efficient nuclear import in the lung airways of the mouse model following intratracheal administration (N=6 mice per group). A transgenic mouse model of MOLDs exhibiting more pronounced airway mucus barrier (i.e., Scnn1b-Tg mice; (10, 79)) will be used, thereby implementing more stringent criteria potentially for broader utility of finally selected candidates. Scnn1b-Tg mice at the age of 4 weeks will intratracheally receive a library of enriched mAAV6, and one week after the administration, lung tissues will be thoroughly washed via bronchoalveolar lavage to remove mucus-associated mAAV6, and CD45-negative cells will be harvested (73). Subsequently, lead candidates will be then selected using the method described herein. To evaluate the effect of pre-existing NAb against wild-type AAV6, a separate group of animals will intratracheally receive wild-type AAV6 as described above, 2 weeks prior to the administration of the enriched mAAV6 library. It is expected that 6 lead candidates that exhibit greatest accumulation of self-encoding DNA cassettes in cell nuclei of lung airway cells while satisfying the selection criterion for human airway mucus penetration will be selected. The lead mutants selected here may or may not overlap with those identified for primary HAEs. If the lead candidates are sharply distinct between two species (i.e., human versus mouse), subsequent studies using respective lead mAAV6 for the ALI cultures and the mouse model will be pursued.

Assessing the coverage and overall level of reporter transgene expression by EVmAAV6 prepared with selected mutants in vitro and in vivo. Determine the percentages of transduced cells and the overall levels of reporter transgene expression by EVmAAV6 prepared with mAAV6 selected in (A) ALI cultures of human primary airway cells and (B) the lungs of the mucus-plugged mouse model. (C) Investigate the ability of EVmAAV6 to deliver two reporter cassettes individually packaged in mAAV6 into the same cells for potential delivery of large genes beyond the natural AAV packaging capacity.

EVmAAV6, will be prepared based on 6 lead mAAV6 candidates and confirmed for ex vivo mucus penetration and human airway mucus using MPT. Reporter transgene expression will be evaluated in ALI cultures in vitro. Briefly, after transfection of HEK293 cells with packaging plasmids, including ITP-containing plasmid carrying YFP and luciferase co-expressing cassette, cells and supernatants will be harvested. While standard (i.e., EV-free) mAAV6 will be obtained from cells using a standard procedure, respective EVmAAV6 will be collected from the supernatant via sequential centrifugation at 100,000×g and filtration with a 0.45-μm cellulose acetate filter. EVmAAV6 will then be characterized for structure, EV concentration and size (i.e., hydrodynamic diameter) using TEM, a Spectradyne particle analyzer and a Malvern Zetasizer (i.e., DLS). In parallel, EVmAAV6 will be screened for common EV biomarkers by single-particle interferometric reflectance imaging sensing with fluorescence using tetraspanin chips. AAV titers of mAAV6 and EVmAAV6 will be analyzed by quantitative PCR. EVs will also be harvested from untransfected HEK293 cells for preparation of post-mixtures of EVs and lead mAAV6 candidates (i.e., EV+mAAV6). EV-free mAAV6, EVmAAV6 or respective EV+mAAV6 will be administered to the apical surface of the mucus-covered ALI culture, and cells will be then harvested two days after the administration, and a half of the harvested cells will be evaluated for the percentage of YFP-positive cells that indicates the coverage of transgene expression, using confocal microscopy and flow cytometry. The rest of cells will be used for homogenate-based luciferase assay to determine the overall level of transgene expression. Three different versions (i.e., EV-free, EV-associated and post-mixture) of wild-type AAV6 will also be prepared and tested for comparison.

EVmAAV6 will be prepared based on 6 lead mAAV6 candidates that have been confirmed for ex vivo mucus penetration. The ability of these EVmAAV6 to penetrate human airway mucus using MPT, will be first confirmed. Distribution and overall level of transgene expression in the mucus-plugged lungs of the transgenic mouse model will be evaluated (i.e., Scnn1b-Tg mice) (N=6 mice per group). A pair of AAV6 and EVAAV6 will also be prepared for comparisons with lead mutant pairs. Each animal will intratracheally receive either AAV6/mAAV6 or EVAAV6/EVmAAV6 carrying the above-mentioned dual reporter co-expressing cassette, and lung tissue will be harvested 2 weeks after the administration. Lung tissues will be harvested to assess the percentage of transduced cells or coverage using flow cytometry or confocal microscopy, respectively, as we routinely conduct (10, 73). In parallel, lung tissues will be harvested from separate sets of transduced animals and the overall level of transgene expression will be evaluated by in vivo imaging with an In Vivo Imaging System (IVIS), followed by homogenate-based luciferase assay. One EVmAAV6 each exhibiting either greatest distribution or overall levels of reporter transgene expression in the mouse lungs (so up to 2 lead EVmAAV6) will be determined for further evaluation.

The potential of using EVmAAV6 for delivery of a large gene beyond the natural AAV packaging capacity (i.e., 4.7 kb) will be evaluated. Dual AAV vectors carrying either upstream or downstream transgene sequence have been used to deliver of large genes (98-100), but such approaches often lead to poor transgene expression as simultaneous delivery of dual vectors to the same cells is not readily achieved. On the other hand, it is hypothesized that two or more AAV vectors, individually carrying different DNA cassettes, co-associated with the same EVs will likely enhance the probability of simultaneous delivery and thus the production of large therapeutic proteins. To test this hypothesis, a pair of lead mAAV6 and EVmAAV6, will be prepared with two different ITR-containing plasmids carrying either ZsGreen- or mCherry-expressing DNA cassette and assess their ability to mediate dual reporter expression in the same cells in vitro and in vivo (N=6 mice per group). Treatment will be executed as described and efficacy of the same-cell dual reporter transgene expression will be assessed using confocal microscopy and/or flow cytometry.

Example 3: Evaluation of EVAAV6 and lead EVmAAV6 to provide sustained pulmonary transgene expression. (A) Evaluate the ability to transduce human primary airway progenitor cells (i.e., basal cells) and ALI cultures of human primary airway cells after incubation in AAV6-seropositive human serum. (B) Determine the humoral and cell-mediated immune responses against AAV6 in vivo. (C) Assess the ability to retain the transduction efficacy in the lung in vivo (i) after incubation in AAV6-seropositive human serum or (ii) upon repeated dosing.

The in vitro primary ALI culture transduction experiments will be conducted after pretreating EVAAV6 and lead EVmAAV6 with serum from wild-type AAV6-seropositive patients. The level of transgene expression in comparison to the outcomes without serum incubation will be used as a readout confirming the escape from neutralization. In parallel, the ability to transduce p63- and cytokeratin 5-positive human primary airway basal cells will be assessed using undifferentiated primary human airway cells.

Humoral and cell-mediated immune responses as well as vector associated inflammation will be evaluated using C57BL/6 mice that serves as a background strain for mouse models of MOLDs (i.e., Scnn1b-Tg mice; Aim 1 and 2) and allergic asthma. The acute inflammation associated with activation of TLR9/MyD88 will be assessed by Multiplex Bead Array Assay for detection of 25 soluble pro-inflammatory cytokines/chemokines (GM-CSF, IL-1β, IL-6, IL-2, IL-2R, IL-4, IL-5, IL-10, and MCP-1, etc.) on serum samples collected 2, 8 and 24 hours (short-term) and 7, 14, 21 and 28 days (long-term) after the intratracheal administration of EVAAV6 or lead EVmAAV6 (N=6 mice per group). The level of neutralizing activity of AAV-specific NAbs will be evaluated by transduction inhibition-based assay (102). Cellular immune responses will be measured by interferon gamma (IFNγ/TNFα) ELISpot assay for both CD8⁺ and CD4⁺ on cells isolated from splenocytes with AAV6 capsid dominant peptides (103).

Immunodeficient NOD SCID gamma (NSG) mice will be intratracheally treated with EVAAV6 or lead EVmAAV6, following intravenous infusion of immunoglobulins from wild-type AAV6-seropositive patients at escalating doses of 5, 50, and 500 mg/kg (N=6 mice per group). The inhibitory effect or escape from neutralization will be evaluated by assessing the overall luciferase transgene expression level in the mouse lungs by live animal imaging and tissue homogenate-based luciferase assay. Using C57BL/6 mice, we will also evaluate the possibility to overcome AAV-mediated humoral immunity by repeating the dosing of EVAAV6 or lead EVmAAV6 carrying different reporter genes (i.e., mCherry and luciferase for the first and second doses, respectively, to make comparisons with the single dose study) at different time intervals, including 1, 3 and 5 month(s), with varying degrees of neutralizing activity if any (N=6 mice per group). In parallel, the level of AAV-specific NAb will be evaluated 1, 3 and 5 month(s) after the initial administration.

Example 4: Safety and therapeutic efficacy of inhaled thymulin gene therapy by EVAAV6 and lead EVmAAV6 in vivo. (A) Determine the doses providing the greatest thymulin transgene expression in the lung without incurring adverse effects using healthy mice. (B) Assess the therapeutic efficacy of the inhaled thymulin gene therapy using mouse models of allergic asthma.

A dose escalation study will be conducted where three incrementing doses in the range of 4×10⁹-1×10¹² will be tested (N=6 mice per group). Specifically, EVAAV6 and up to two lead EVmAAV6 will be constructed to carry a thymulin-expressing cassette and will be administered intratracheally into the lungs of C57BL/6 mice. Triplicate sets of animals will be used to evaluate thymulin transgene expression and local and systemic safety profiles. Lungs of the animals in the first set will be harvested in 2 weeks after the administration, followed by the quantification of the level of thymulin transgene expression by ELISA. Animals in the other two sets will be subjected to short- and long-term safety assessment. Specifically, animals will be sacrificed 24 hours and 2 weeks after the administration and lung tissues and whole blood samples will be harvested. Lung tissues will then be subjected to quantification of pro-inflammatory mediators, including but not limited to NF-κB, TRL9, MyD88, IL-1β, IL-6 and IL-12, covered by a commercially available quantitative RT-PCR array. In parallel, blood samples collected from the animals will be used to conduct blood biochemistry analysis. Based on the findings here, will determine optimal doses for EVAAV6 and lead EVmAAV6 that provide maximal thymulin transgene expression in the lung without eliciting local and systemic adverse effects, which will be employed in the preclinical thymulin gene therapy.

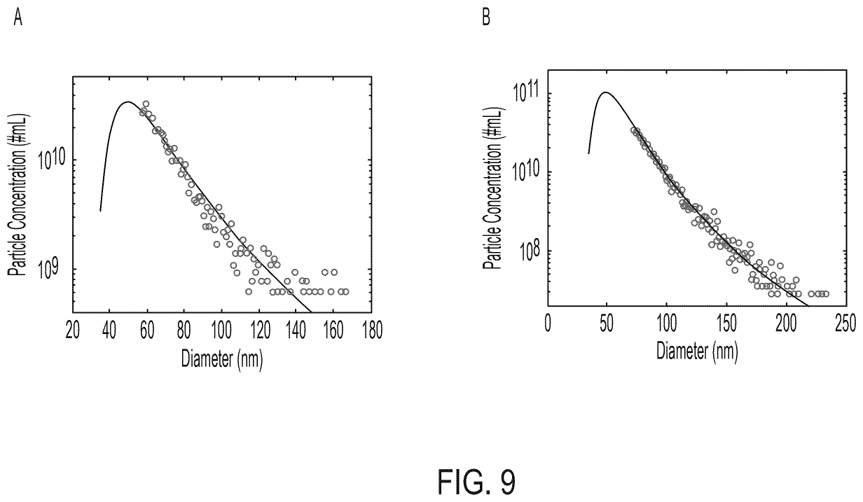

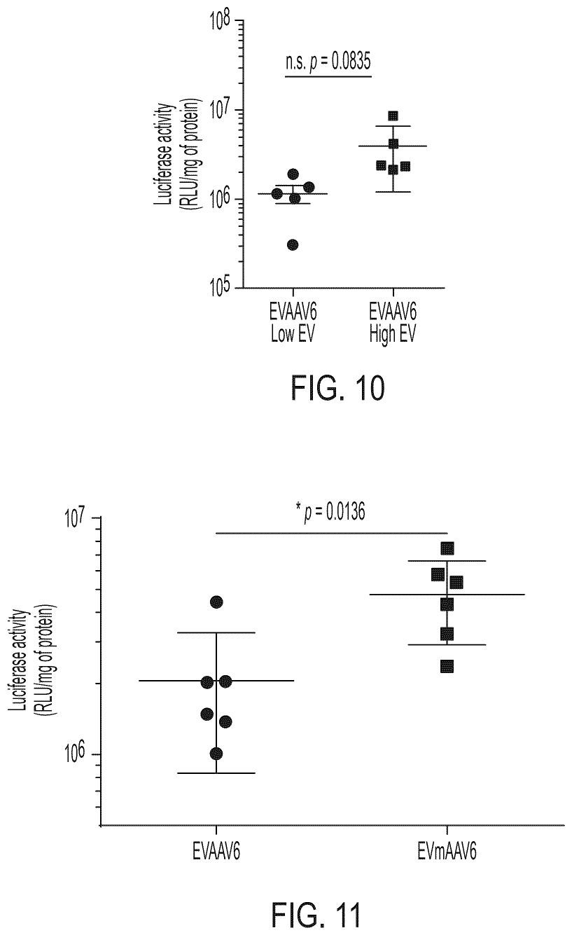

The immunomodulatory and therapeutic effects will be investigated in two mouse models of allergic asthma established on C57BL/6 mice, following intratracheal administration of EVAAV6 or lead EVmAAV6 carrying a thymulin-expressing cassette, at respective optimal doses determined in Aim 3A (N=8 mice per group). OVA-based model will be established by a series of sequential intraperitoneal sensitization (7 times every other day starting on Day 1) and intratracheal challenge (3 times every third day starting on Day 40) with OVA at 10 and 20 μg, respectively (74). HDM-based model will be established by intranasal instillation of 25 μg HDM (i.e., Dermatophagoides pteronyssinus) 5 days a week for 4 weeks (77). For the OVA-based model, animals will be treated on Day 47 and efficacy will be evaluated on Day 67 from the initial sensitization, based on key disease phenotypes that are fully established and retained without self-resolution during this time window (74). For the HDM-based model, all key disease phenotypes are established by the end of the 4-week 5-day weekly HDM instillation and lasted up 8^{th} week with minor increases in asthmatic inflammation and pulmonary fibrosis upon a continuous HDM instillation (77). Animals will be treated at the beginning of 5^{th} week and efficacy evaluated at the end of 8^{th} week. Live animals will be subjected to methacholine challenge test to determine AHR (i.e., airway resistance and dynamic compliance), as shown in FIG. 9. Animals will then be subjected to comprehensive assessments of immunomodulatory and therapeutic efficacy assessments. Histopathological analysis will be conducted to assess the goblet cell metaplasia (AB-PAS staining), lung inflammation (H&E), airway constriction (H&E; contraction index) and collagen deposition (Masson's trichrome staining). Using a duplicate set of animals, BALF and/or whole lung tissues will be collected to evaluate immune infiltration (total and differential cell counting) and levels of soluble factors using ELISA as shown in FIG. 8. Those include Th2 cytokines (IL-4 and IL-13), an anti-inflammatory cytokine (IL-10), pro-fibrotic/remodeling mediators (VEGF and TGF-β) and immune cell-recruiting chemokines (eosinophil: CCL11; neutrophil: CXCL1; macrophage: CCL5; Treg: CCL17). In parallel, phenotypic changes of T cells (Th2 vs. Treg) and macrophages (M1 vs. M2) will be evaluated by the disease and therapeutic intervention (by thymulin gene therapy) using RT-PCR (74).

### REFERENCES

1. Maas S L N, Breakefield X O, Weaver A M. Extracellular Vesicles: Unique Intercellular Delivery Vehicles. Trends Cell Biol. 2017; 27(3):172-88. Epub 2016/12/17. doi: 10.1016/j.tcb.2016.11.003. PubMed PMID: 27979573; PMCID: PMC5318253.
2. Nam G H, Choi Y, Kim G B, Kim S, Kim S A, Kim I S. Emerging Prospects of Exosomes for Cancer Treatment: From Conventional Therapy to Immunotherapy. Adv Mater. 2020; 32(51):e2002440. Epub 2020/10/06. doi: 10.1002/adma.202002440. PubMed PMID: 33015883.
3. Mishra A, Singh P, Qayoom I, Prasad A, Kumar A. Current strategies in tailoring methods for engineered exosomes and future avenues in biomedical applications. J Mater Chem B. 2021; 9(32):6281-309. Epub 2021/07/22. doi: 10.1039/d1tb01088c. PubMed PMID: 34286815.
4. Gyorgy B, Maguire C A. Extracellular vesicles: nature's nanoparticles for improving gene transfer with adeno-associated virus vectors. Wiley Interdiscip Rev Nanomed Nanobiotechnol. 2018; 10(3):e1488. Epub 2017/08/12. doi: 10.1002/wnan.1488. PubMed PMID: 28799250.
5. Hudry E, Martin C, Gandhi S, Gyorgy B, Scheffer D I, Mu D, Merkel S F, Mingozzi F, Fitzpatrick Z, Dimant H, Masek M, Ragan T, Tan S, Brisson A R, Ramirez S H, Hyman B T, Maguire C A. Exosome-associated AAV vector as a robust and convenient neuroscience tool. Gene Ther. 2016; 23(4):380-92. Epub 2016/02/03. doi: 10.1038/gt.2016.11. PubMed PMID: 26836117; PMCID: PMC4824662.
6. Wassmer S J, Carvalho L S, Gyorgy B, Vandenberghe L H, Maguire C A. Exosome-associated AAV2 vector mediates robust gene delivery into the murine retina upon intravitreal injection. Sci Rep. 2017; 7:45329. Epub 2017/04/01. doi: 10.1038/srep45329. PubMed PMID: 28361998; PMCID: PMC5374486.
7. Liu B, Li Z, Huang S, Yan B, He S, Chen F, Liang Y. AAV-Containing Exosomes as a Novel Vector for Improved Gene Delivery to Lung Cancer Cells. Front Cell Dev Biol. 2021; 9:707607. Epub 2021/09/07. doi: 10.3389/fcell.2021.707607. PubMed PMID: 34485293; PMCID: PMC8414974.
8. Gyorgy B, Sage C, Indzhykulian A A, Scheffer D I, Brisson A R, Tan S, Wu X, Volak A, Mu D, Tamvakologos P I, Li Y, Fitzpatrick Z, Ericsson M, Breakefield X O, Corey D P, Maguire C A. Rescue of Hearing by Gene Delivery to Inner-Ear Hair Cells Using Exosome-Associated AAV. Mol Ther. 2017; 25(2):379-91. Epub 2017/01/14. doi: 10.1016/j.ymthe.2016.12.010. PubMed PMID: 28082074; PMCID: PMC5368844.
9. Maguire C A, Balaj L, Sivaraman S, Crommentuijn M H, Ericsson M, Mincheva-Nilsson L, Baranov V, Gianni D, Tannous B A, Sena-Esteves M, Breakefield X O, Skog J. Microvesicle-associated AAV vector as a novel gene delivery system. Mol Ther. 2012; 20(5):960-71. Epub 2012/02/09. doi: 10.1038/mt.2011.303. PubMed PMID: 22314290; PMCID: PMC3345986.
10. Duncan G A, Kim N, Colon-Cortes Y, Rodriguez J, Mazur M, Birket S E, Rowe S M, West N E, Livraghi-Butrico A, Boucher R C, Hanes J, Aslanidi G, Suk J S. An Adeno-Associated Viral Vector Capable of Penetrating the Mucus Barrier to Inhaled Gene Therapy. Mol Ther Methods Clin Dev. 2018; 9:296-304. doi: 10.1016/j.omtm.2018.03.006. PubMed PMID: 30038933; PMCID: PMC6054694.
11. Wang Y Y, Harit D, Subramani D B, Arora H, Kumar P A, Lai S K. Influenza-binding antibodies immobilise influenza viruses in fresh human airway mucus. Eur Respir J. 2017; 49(1). doi: 10.1183/13993003.01709-2016. PubMed PMID: 28122865.
12. Schuster B S, Kim A J, Kay J C, Kanzawa M M, Guggino W B, Boyle M P, Rowe S M, Muzyczka N, Suk J S, Hanes J. Overcoming the cystic fibrosis barrier to leading adeno-associated virus gene therapy vectors. Mol Ther. 2014; in press.
13. Jensen M A, Wang Y Y, Lai S K, Forest M G, McKinley S A. Antibody-Mediated Immobilization of Virions in Mucus. Bull Math Biol. 2019; 81(10):4069-99. doi: 10.1007/s11538-019-00653-6. PubMed PMID: 31468263; PMCID: PMC6764938.
14. Gyorgy B, Fitzpatrick Z, Crommentuijn M H, Mu D, Maguire C A. Naturally enveloped AAV vectors for shielding neutralizing antibodies and robust gene delivery in vivo. Biomaterials. 2014; 35(26):7598-609. Epub 2014/06/12. doi: 10.1016/j.biomaterials.2014.05.032. PubMed PMID: 24917028; PMCID: PMC4104587.
15. Mingozzi F, High K A. Immune responses to AAV vectors: overcoming barriers to successful gene therapy. Blood. 2013; 122(1):23-36. Epub 2013/04/19. doi: 10.1182/blood-2013-01-306647. PubMed PMID: 23596044; PMCID: PMC3701904.
16. High K A, Aubourg P. rAAV human trial experience. Methods in molecular biology (Clifton, NJ. 2011; 807:429-57. PubMed PMID: 22034041.
17. Mendell J R, Rodino-Klapac L, Sahenk Z, Malik V, Kaspar B K, Walker C M, Clark K R. Gene therapy for muscular dystrophy: Lessons learned and path forward. Neuroscience letters. 2012. PubMed PMID: 22609847.
18. Jacobson S G, Cideciyan A V, Roman A J, Sumaroka A, Schwartz S B, Heon E, Hauswirth W W. Improvement and decline in vision with gene therapy in childhood blindness. N Engl J Med. 2015; 372(20):1920-6. Epub 2015/05/06. doi: 10.1056/NEJMoa1412965. PubMed PMID: 25936984; PMCID: PMC4450362.
19. Flotte T R, Mueller C. Gene therapy for alpha-1 antitrypsin deficiency. Human molecular genetics. 2012; 20(R1):R87-92. PubMed PMID: 21498872.
20. Smalley E. First AAV gene therapy poised for landmark approval. Nat Biotechnol. 2017; 35(11):998-9. Epub 2017/11/10. doi: 10.1038/nbt1117-998. PubMed PMID: 29121014.
21. Keeler A M, Flotte T R. Recombinant Adeno-Associated Virus Gene Therapy in Light of Luxturna (and Zolgensma and Glybera): Where Are We, and How Did We Get Here? Annu Rev Virol. 2019. Epub 2019/07/10. doi: 10.1146/annurev-virology-092818-015530. PubMed PMID: 31283441.
22. Cooney A L, McCray P B, Jr., Sinn P L. Cystic Fibrosis Gene Therapy: Looking Back, Looking Forward. Genes (Basel). 2018; 9(11). Epub 2018/11/09. doi: 10.3390/genes9110538. PubMed PMID: 30405068; PMCID: PMC6266271.
23. Kim N, Duncan G A, Hanes J, Suk J S. Barriers to inhaled gene therapy of obstructive lung diseases: A review. J Control Release. 2016; 240:465-88. Epub 2016/05/20. doi: 10.1016/j.jconrel.2016.05.031. PubMed PMID: 27196742; PMCID: PMC5064827.
24. Griesenbach U, Alton E W, Consortium UKCFGT. Gene transfer to the lung: lessons learned from more than 2 decades of CF gene therapy. Adv Drug Deliv Rev. 2009; 61(2):128-39. doi: 10.1016/j.addr.2008.09.010. PubMed PMID: 19138713.
25. Guggino W B, Cebotaru L. Adeno-Associated Virus (AAV) gene therapy for cystic fibrosis: current barriers and recent developments. Expert Opin Biol Ther. 2017; 17(10):1265-73. Epub 2017/06/29. doi: 10.1080/14712598.2017.1347630. PubMed PMID: 28657358; PMCID: PMC5858933.
26. Yan Z, McCray P B, Jr., Engelhardt J F. Advances In Gene Therapy For Cystic Fibrosis Lung Disease. Human molecular genetics. 2019. Epub 2019/07/25. doi: 10.1093/hmg/ddz139. PubMed PMID: 31332440.
27. van Haasteren J, Hyde S C, Gill D R. Lessons learned from lung and liver in-vivo gene therapy: implications for the future. Expert Opin Biol Ther. 2018; 18(9):959-72. Epub 2018/08/02. doi: 10.1080/14712598.2018.1506761. PubMed PMID: 30067117; PMCID: PMC6134476.
28. Cone R A. Barrier properties of mucus. Adv Drug Deliv Rev. 2009; 61:75-85. doi: 10.1016/j.addr.2008.09.008. PubMed PMID: 19135107.
29. Duncan G A, Jung J, Hanes J, Suk J S. The Mucus Barrier to Inhaled Gene Therapy. Mol Ther. 2016; 24(12):2043-53. Epub 2016/11/02. doi: 10.1038/mt.2016.182. PubMed PMID: 27646604; PMCID: PMC5167788.
30. Ferrari S, Geddes D M, Alton E W. Barriers to and new approaches for gene therapy and gene delivery in cystic fibrosis. Adv Drug Deliv Rev. 2002; 54(11):1373-93. Epub 2002/11/30. doi: 10.1016/S0169-409X(02)00145-X. PubMed PMID: 12458150.
31. Kim N, Duncan G A, Hanes J, Suk J S. Barriers to inhaled gene therapy of obstructive lung diseases: A review. Journal of Controlled Release. 2016; 240:465-88.
32. Mastorakos P, da Silva A L, Chisholm J, Song E, Choi W K, Boyle M P, Morales M M, Hanes J, Suk J S. Highly compacted biodegradable DNA nanoparticles capable of overcoming the mucus barrier for inhaled lung gene therapy. Proc Natl Acad Sci USA. 2015; 112(28):8720-5. Epub 2015/07/01. doi: 10.1073/pnas.1502281112. PubMed PMID: 26124127; PMCID: 4507234.
33. Schneider C S, Xu Q, Boylan N J, Chisholm J, Tang B C, Schuster B S, Henning A, Ensign L M, Lee E, Adstamongkonkul P, Simons B W, Wang S S, Gong X, Yu T, Boyle M P, Suk J S, Hanes J. Nanoparticles that do not adhere to mucus provide uniform and long-lasting drug delivery to airways following inhalation. Sci Adv. 2017; 3(4):e1601556. Epub 2017/04/25. doi: 10.1126/sciadv.1601556. PubMed PMID: 28435870; PMCID: 5381952.
34. Suk J S, Kim A J, Trehan K, Schneider C S, Cebotaru L, Woodward O M, Boylan N J, Boyle M P, Lai S K, Guggino W B, Hanes J. Lung gene therapy with highly compacted DNA nanoparticles that overcome the mucus barrier. Journal of Controlled Release. 2014; 178:8-17. Epub 2014/01/21. doi: 10.1016/j.jconrel.2014.01.007. PubMed PMID: 24440664; PMCID: 3951606.
35. Suk J S. Could recent advances in DNA-loaded nanoparticles lead to effective inhaled gene therapies? Nanomedicine (Lond). 2016. Epub 2016/01/20. doi: 10.2217/nnm.15.194. PubMed PMID: 26783765.
36. Forier K, Messiaen A S, Raemdonck K, Deschout H, Rejman J, De Baets F, Nelis H, De Smedt S C, Demeester J, Coenye T, Braeckmans K. Transport of nanoparticles in cystic fibrosis sputum and bacterial biofilms by single-particle tracking microscopy. Nanomedicine (Lond). 2013; 8(6):935-49. Epub 2012/10/06. doi: 10.2217/nnm.12.129. PubMed PMID: 23035662
37. Chen D, Liu J, Wu J, Suk J S. Enhancing nanoparticle penetration through airway mucus to improve drug delivery efficacy in the lung. Expert Opin Drug Deliv. 2020:1-12. Epub 2020/11/22. doi: 10.1080/17425247.2021.1854222. PubMed PMID: 33218265.
38. Carneiro A, Lee H, Lin L, van Haasteren J, Schaffer D V. Novel Lung Tropic Adeno-Associated Virus Capsids for Therapeutic Gene Delivery. Hum Gene Ther. 2020; 31(17-18):996-1009. Epub 2020/08/18. doi: 10.1089/hum.2020.169. PubMed PMID: 32799685.
39. Cone R A. Barrier properties of mucus. Adv Drug Deliv Rev. 2009; 61(2):75-85. Epub 2009/01/13. doi: S0169-409X(08)00259-7 [pii]10.1016/j.addr.2008.09.008. PubMed PMID: 19135107.
40. Chisholm J F, Shenoy S K, Shade J K, Kim V, Putcha N, Carson K A, Wise R, Hansel N N, Hanes J S, Suk J S, Neptune E. Nanoparticle diffusion in spontaneously expectorated sputum as a biophysical tool to probe disease severity in COPD. Eur Respir J. 2019; 54(2). Epub 2019/06/06. doi: 10.1183/13993003.00088-2019. PubMed PMID: 31164433; PMCID: PMC8081045.
41. Morgan L E, Jaramillo A M, Shenoy S K, Raclawska D, Emezienna N A, Richardson V L, Hara N, Harder A Q, NeeDell JC, Hennessy C E, El-Batal H M, Magin C M, Grove Villalon D E, Duncan G, Hanes J S, Suk J S, Thornton D J, Holguin F, Janssen W J, Thelin W R, Evans C M. Disulfide disruption reverses mucus dysfunction in allergic airway disease. Nat Commun. 2021; 12(1):249. Epub 2021/01/13. doi: 10.1038/s41467-020-20499-0. PubMed PMID: 33431872; PMCID: PMC7801631.
42. Hida K, Lai S K, Suk J S, Won S Y, Boyle M P, Hanes J. Common gene therapy viral vectors do not efficiently penetrate sputum from cystic fibrosis patients. PloS one. 2011; 6(5):e19919. Epub 2011/06/04. doi: 10.1371/journal.pone.0019919. PubMed PMID: 21637751; PMCID: 3103503.
43. Boylan N J, Suk J S, Lai S K, Jelinek R, Boyle M P, Cooper M J, Hanes J. Highly compacted DNA nanoparticles with low MW PEG coatings: In vitro, ex vivo and in vivo evaluation. J Control Release. 2011; 157(1):72-9. Epub 2011/09/10. doi: 50168-3659(11)00643-2 [pii]10.1016/j.jconrel.2011.08.031. PubMed PMID: 21903145.
44. Suk J S, Boylan N J, Trehan K, Tang B C, Schneider C S, Lin J M, Boyle M P, Zeitlin P L, Lai S K, Cooper M J, Hanes J. N-acetylcysteine enhances cystic fibrosis sputum penetration and airway gene transfer by highly compacted DNA nanoparticles. Mol Ther. 2011; 19(11):1981-9. Epub 2011/08/11. doi: mt2011160 [pii]10.1038/mt.2011.160. PubMedPMID: 21829177.
45. Liu X, Luo M, Trygg C, Yan Z, Lei-Butters D C, Smith C I, Fischer A C, Munson K, Guggino W B, Bunnell B A, Engelhardt J F. Biological differences in rAAV transduction of airway epithelia in humans and in old world non-human primates. Mol Ther. 2007; 15(12):2114-23. Epub 2007/08/02. doi: 10.1038/sj.mt.6300277. PubMed PMID: 17667945; PMCID: 2121582.
46. Liu X, Luo M, Guo C, Yan Z, Wang Y, Engelhardt J. Comparative biology of rAAV transduction in ferret, pig and human airway epithelia. Gene Therapy. 2007; 14(21):1543.
47. Wu Z, Miller E, Agbandje-McKenna M, Samulski R J. Alpha2,3 and alpha2,6 N-linked sialic acids facilitate efficient binding and transduction by adeno-associated virus types 1 and 6. J Virol. 2006; 80(18):9093-103. Epub 2006/08/31. doi: 10.1128/JVI.00895-06. PubMed PMID: 16940521; PMCID: PMC1563919.
48. Wu N H, Yang W, Beineke A, Dijkman R, Matrosovich M, Baumgartner W, Thiel V, Valentin-Weigand P, Meng F, Herrler G. The differentiated airway epithelium infected by influenza viruses maintains the barrier function despite a dramatic loss of ciliated cells. Sci Rep. 2016; 6:39668. Epub 2016/12/23. doi: 10.1038/srep39668. PubMed PMID: 28004801; PMCID: PMC5177954.
49. Ding K, Shen J, Hackett S, Khan M, Campochiaro P A. Proteosomal degradation impairs transcytosis of AAV vectors from suprachoroidal space to retina. Gene Ther. 2021. Epub 2021/02/06. doi: 10.1038/s41434-021-00233-1. PubMed PMID: 33542456; PMCID: PMC8333227.
50. Monahan P E, Lothrop C D, Sun J, Hirsch M L, Kafri T, Kantor B, Sarkar R, Tillson D M, Elia J R, Samulski R J. Proteasome inhibitors enhance gene delivery by AAV virus vectors expressing large genomes in hemophilia mouse and dog models: a strategy for broad clinical application. Mol Ther. 2010; 18(11):1907-16. Epub 2010/08/12. doi: 10.1038/mt.2010.170. PubMed PMID: 20700109; PMCID: PMC2990516.
51. Zhong L, Li B, Jayandharan G, Mah C S, Govindasamy L, Agbandje-McKenna M, Herzog R W, Weigel-Van Aken K A, Hobbs J A, Zolotukhin S, Muzyczka N, Srivastava A. Tyrosine-phosphorylation of AAV2 vectors and its consequences on viral intracellular trafficking and transgene expression. Virology. 2008; 381(2):194-202. Epub 2008/10/07. doi: 10.1016/j.virol.2008.08.027. PubMed PMID: 18834608; PMCID: PMC2643069.
52. Dhungel B P, Bailey C G, Rasko J E J. Journey to the Center of the Cell: Tracing the Path of AAV Transduction. Trends Mol Med. 2021; 27(2):172-84. Epub 2020/10/20. doi: 10.1016/j.molmed.2020.09.010. PubMed PMID: 33071047.
53. Cooney A L, Thornell I M, Singh B K, Shah V S, Stoltz D A, McCray P B, Jr., Zabner J, Sinn P L. A Novel AAV-mediated Gene Delivery System Corrects CFTR Function in Pigs. Am J Respir Cell Mol Biol. 2019; 61(6):747-54. Epub 2019/06/12. doi: 10.1165/rcmb.2019-00060C. PubMed PMID: 31184507; PMCID: PMC6890402.
54. Nidetz N F, McGee M C, Tse L V, Li C, Cong L, Li Y, Huang W. Adeno-associated viral vector-mediated immune responses: Understanding barriers to gene delivery. Pharmacol Ther. 2020; 207:107453. Epub 2019/12/15. doi: 10.1016/j.pharmthera.2019.107453. PubMed PMID: 31836454; PMCID: PMC6980784.
55. Riyad J M, Weber T. Intracellular trafficking of adeno-associated virus (AAV) vectors: challenges and future directions. Gene Ther. 2021. Epub 2021/03/05. doi: 10.1038/s41434-021-00243-z. PubMed PMID: 33658649; PMCID: PMC8413391.
56. Naso M F, Tomkowicz B, Perry W L, 3rd, Strohl W R. Adeno-Associated Virus (AAV) as a Vector for Gene Therapy. BioDrugs. 2017; 31(4):317-34. Epub 2017/07/03. doi: 10.1007/s40259-017-0234-5. PubMed PMID: 28669112; PMCID: PMC5548848.
57. Li C, He Y, Nicolson S, Hirsch M, Weinberg M S, Zhang P, Kafri T, Samulski R J. Adeno-associated virus capsid antigen presentation is dependent on endosomal escape. J Clin Invest. 2013; 123(3):1390-401. Epub 2013/03/05. doi: 10.1172/JC166611. PubMed PMID: 23454772; PMCID: PMC3582142.
58. Karman J, Gumlaw N K, Zhang J, Jiang J L, Cheng S H, Zhu Y. Proteasome inhibition is partially effective in attenuating pre-existing immunity against recombinant adeno-associated viral vectors. PloS one. 2012; 7(4):e34684. Epub 2012/04/20. doi: 10.1371/journal.pone.0034684. PubMed PMID: 22514654; PMCID: PMC3326043.
59. Buning H, Srivastava A. Capsid Modifications for Targeting and Improving the Efficacy of AAV Vectors. Mol Ther Methods Clin Dev. 2019; 12:248-65. Epub 2019/03/01. doi: 10.1016/j.omtm.2019.01.008. PubMed PMID: 30815511; PMCID: PMC6378346.
60. Li B, Ma W, Ling C, Van Vliet K, Huang L Y, Agbandje-McKenna M, Srivastava A, Aslanidi G V. Site-Directed Mutagenesis of Surface-Exposed Lysine Residues Leads to Improved Transduction by AAV2, But Not AAV8, Vectors in Murine Hepatocytes In Vivo. Hum Gene Ther Methods. 2015; 26(6):211-20. Epub 2015/10/01. doi: 10.1089/hgtb.2015.115. PubMed PMID: 26421998; PMCID: PMC4677520.
61. Markusic D M, Herzog R W, Aslanidi G V, Hoffman B E, Li B, Li M, Jayandharan G R, Ling C, Zolotukhin I, Ma W, Zolotukhin S, Srivastava A, Zhong L. High-efficiency transduction and correction of murine hemophilia B using AAV2 vectors devoid of multiple surface-exposed tyrosines. Mol Ther. 2010; 18(12):2048-56. Epub 2010/08/26. doi: mt2010172 [pii]10.1038/mt.2010.172. PubMed PMID: 20736929; PMCID: 2997584.
62. Aslanidi G V, Rivers A E, Ortiz L, Song L, Ling C, Govindasamy L, Van Vliet K, Tan M, Agbandje-McKenna M, Srivastava A. Optimization of the capsid of recombinant adeno-associated virus 2 (AAV2) vectors: the final threshold? PloS one. 2013; 8(3):e59142. Epub 2013/03/26. doi: 10.1371/journal.pone.0059142 PONE-D-12-28292 [pii]. PubMed PMID: 23527116; PMCID: 3602601.
63. Pandya J, Ortiz L, Ling C, Rivers A E, Aslanidi G. Rationally designed capsid and transgene cassette of AAV6 vectors for dendritic cell-based cancer immunotherapy. Immunol Cell Biol. 2014; 92(2):116-23. doi: 10.1038/icb.2013.74. PubMed PMID: 24217810.
64. Ling C, Wang Y, Lu Y, Wang L, Jayandharan G R, Aslanidi G V, Li B, Cheng B, Ma W, Lentz T, Ling C, Xiao X, Samulski R J, Muzyczka N, Srivastava A. Enhanced transgene expression from recombinant single-stranded D-sequence-substituted adeno-associated virus vectors in human cell lines in vitro and in murine hepatocytes in vivo. J Virol. 2015; 89(2):952-61. doi: 10.1128/JVI.02581-14. PubMed PMID: 25355884; PMCID: PMC4300666.
65. Pandya M, Britt K, Hoffman B, Ling C, Aslanidi G V. Reprogramming Immune Response With Capsid-Optimized AAV6 Vectors for Immunotherapy of Cancer. J Immunother. 2015; 38(7):292-8. Epub 2015/08/12. doi: 10.1097/CJI.0000000000000093 00002371-201509000-00005 [pii]. PubMed PMID: 26261893; PMCID: 4535186.
66. Ling C, Yin Z, Li J, Zhang D, Aslanidi G, Srivastava A. Strategies to generate high-titer, high-potency recombinant AAV3 serotype vectors. Mol Ther Methods Clin Dev. 2016; 3:16029. doi: 10.1038/mtm.2016.29. PubMed PMID: 27200382; PMCID: PMC4856060.
67. Chen M, Maeng K, Nawab A, Francois R A, Bray J K, Reinhard M K, Boye S L, Hauswirth W W, Kaye F J, Aslanidi G, Srivastava A, Zajac-Kaye M. Efficient Gene Delivery and Expression in Pancreas and Pancreatic Tumors by Capsid-Optimized AAV8 Vectors. Hum Gene Ther Methods. 2017; 28(1):49-59. doi: 10.1089/hgtb.2016.089. PubMed PMID: 28125909; PMCID: PMC5314986.
68. Pandya J, Ortiz L, Ling C, Rivers A E, Aslanidi G. Rationally designed capsid and transgene cassette of AAV6 vectors for dendritic cell-based cancer immunotherapy. Immunol Cell Biol. 2013. Epub 2013/11/13. doi: 10.1038/icb.2013.74 icb201374 [pii]. PubMed PMID: 24217810.
69. Sayroo R, Nolasco D, Yin Z, Colon-Cortes Y, Pandya M, Ling C, Aslanidi G. Development of novel AAV serotype 6 based vectors with selective tropism for human cancer cells. Gene Ther. 2015. Epub 2015/08/14. doi: 10.1038/gt.2015.89 gt201589 [pii]. PubMed PMID: 26270885.
70. McCraw D M, O'Donnell J K, Taylor K A, Stagg S M, Chapman M S. Structure of adeno-associated virus-2 in complex with neutralizing monoclonal antibody A20. Virology. 2012; 431(1-2):40-9. Epub 2012/06/12. doi: 10.1016/j.virol.2012.05.004. PubMed PMID: 22682774; PMCID: PMC3383000.
71. Martino A T, Basner-Tschakarjan E, Markusic D M, Finn J D, Hinderer C, Zhou S, Ostrov D A, Srivastava A, Ertl H C, Terhorst C, High K A, Mingozzi F, Herzog R W. Engineered AAV vector minimizes in vivo targeting of transduced hepatocytes by capsid-specific CD8+ T cells. Blood. 2013; 121(12):2224-33. Epub 2013/01/18. doi: 10.1182/blood-2012-10-460733 blood-2012-10-460733 [pii]. PubMed PMID: 23325831; PMCID: 3606062.
72. Birket S E, Davis J M, Fernandez C M, Tuggle K L, Oden A M, Chu K K, Tearney G J, Fanucchi M V, Sorscher E J, Rowe S M. Development of an airway mucus defect in the cystic fibrosis rat. JCI Insight. 2018; 3(1). Epub 2018/01/13. doi: 10.1172/jci.insight.97199. PubMed PMID: 29321377; PMCID: PMC5821204.
73. Mastorakos P, da Silva A L, Chisholm J, Song E, Choi W K, Boyle M P, Morales M M, Hanes J, Suk J S. Highly compacted biodegradable DNA nanoparticles capable of overcoming the mucus barrier for inhaled lung gene therapy. Proc Natl Acad Sci USA. 2015; 112(28):8720-5. Epub 2015/07/01. doi: 10.1073/pnas.1502281112. PubMed PMTD: 26124127; PMCID: 4507234.
74. da Silva A L, de Oliveira G P, Kim N, Cruz F F, Kitoko J Z, Blanco N G, Martini S V, Hanes J, Rocco P R M, Suk J S, Morales M M. Nanoparticle-based thymulin gene therapy therapeutically reverses key pathology of experimental allergic asthma. Sci Adv. 2020; 6(24):eaay7973. Epub 2020/06/25. doi: 10.1126/sciadv.aay7973. PubMed PMID: 32577505; PMCID: PMC7286682.
75. Piyadasa H, Altieri A, Basu S, Schwartz J, Halayko A J, Mookherjee N. Biosignature for airway inflammation in a house dust mite-challenged murine model of allergic asthma. Biol Open. 2016; 5(2):112-21. doi: 10.1242/bio.014464. PubMed PMID: 26740570; PMCID: PMC4823983.
76. Bracken S J, Adami A J, Szczepanek S M, Ehsan M, Natarajan P, Guernsey L A, Shahriari N, Rafti E, Matson A P, Schramm C M, Thrall R S. Long-Term Exposure to House Dust Mite Leads to the Suppression of Allergic Airway Disease Despite Persistent Lung Inflammation. Int Arch Allergy Immunol. 2015; 166(4):243-58. doi: 10.1159/000381058. PubMed PMID: 25924733; PMCID: PMC4485530.
77. Woo L N, Guo W Y, Wang X, Young A, Salehi S, Hin A, Zhang Y, Scott J A, Chow C W. A 4-Week Model of House Dust Mite (HDM) Induced Allergic Airways Inflammation with Airway Remodeling. Sci Rep. 2018; 8(1):6925. doi: 10.1038/s41598-018-24574-x. PubMed PMID: 29720689; PMCID: PMC5932037.
78. Johnson J R, Pacitto S R, Wong J, Archer E W, Eirefelt S, Miller-Larsson A, Jordana M. Combined budesonide/formoterol therapy in conjunction with allergen avoidance ameliorates house dust mite-induced airway remodeling and dysfunction. Am J Physiol Lung Cell Mol Physiol. 2008; 295(5):L780-8. doi: 10.1152/ajplung.90229.2008. PubMed PMID: 18776055.
79. Kim N, Kwak G, Rodriguez J, Livraghi-Butrico A, Zuo X, Simon V, Han E, Shenoy S K, Pandey N, Mazur M, Birket S E, Kim A, Rowe S M, Boucher R, Hanes J, Suk J S. Inhaled gene therapy of preclinical muco-obstructive lung diseases by nanoparticles capable of breaching the airway mucus barrier. Thorax. 2021. Epub 2021/10/27. doi: 10.1136/thoraxjnl-2020-215185. PubMed PMID: 34697091.
80. Rosario A M, Cruz P E, Ceballos-Diaz C, Strickland M R, Siemienski Z, Pardo M, Schob K L, Li A, Aslanidi G V, Srivastava A, Golde T E, Chakrabarty P. Microglia-specific targeting by novel capsid-modified AAV6 vectors. Mol Ther Methods Clin Dev. 2016; 3:16026. Epub 2016/06/17. doi: 10.1038/mtm.2016.26. PubMed PMID: 27308302; PMCID: PMC4909093.
81. da Silva A L, Martini S V, Abreu S C, Samary Cdos S, Diaz B L, Fernezlian S, de Sa V K, Capelozzi V L, Boylan N J, Goya R G, Suk J S, Rocco P R, Hanes J, Morales M M. DNA nanoparticle-mediated thymulin gene therapy prevents airway remodeling in experimental allergic asthma. J Control Release. 2014; 180:125-33. Epub 2014/02/22. doi: 10.1016/j.jconrel.2014.02.010. PubMed PMID: 24556417; PMCID: PMC3992277.
   Porpodis K, Domvri K, Kontakiotis T, Fouka E, Kontakioti E, Zarogoulidis K, Papakosta D. Comparison of diagnostic validity of mannitol and methacholine challenges and relationship to clinical status and airway inflammation in steroid-naive asthmatic patients. The Journal of asthma: official journal of the Association for the Care of Asthma. 2017; 54(5):520-9. doi: 10.1080/02770903.2016.1238926. PubMed PMID: 27686218.
82. Verhoeven G T, Hegmans J P, Mulder P G, Bogaard J M, Hoogsteden H C, Prins J B. Effects of fluticasone propionate in COPD patients with bronchial hyperresponsiveness. Thorax. 2002; 57(8):694-700. doi: 10.1136/thorax.57.8.694. PubMed PMID: 12149529; PMCID: 1746396.
83. Li C, Wu S, Albright B, Hirsch M, Li W, Tseng Y S, Agbandje-McKenna M, McPhee S, Asokan A, Samulski R J. Development of Patient-specific AAV Vectors After Neutralizing Antibody Selection for Enhanced Muscle Gene Transfer. Mol Ther. 2016; 24(1):53-65. doi: 10.1038/mt.2015.134. PubMed PMID: 26220272; PMCID: PMC4754536.
84. Tse L V, Klinc K A, Madigan V J, Castellanos Rivera R M, Wells L F, Havlik L P, Smith J K, Agbandje-McKenna M, Asokan A. Structure-guided evolution of antigenically distinct adeno-associated virus variants for immune evasion. Proc Natl Acad Sci USA. 2017; 114(24):E4812-E21. doi: 10.1073/pnas.1704766114. PubMed PMID: 28559317; PMCID: PMC5474820.
85. Kotterman M A, Schaffer D V. Engineering adeno-associated viruses for clinical gene therapy. Nat Rev Genet. 2014; 15(7):445-51. doi: 10.1038/nrg3742. PubMed PMID: 24840552; PMCID: PMC4393649.
86. Pekrun K, De Alencastro G, Luo Q J, Liu J, Kim Y, Nygaard S, Galivo F, Zhang F, Song R, Tiffany M R, Xu J, Hebrok M, Grompe M, Kay M A. Using a barcoded AAV capsid library to select for clinically relevant gene therapy vectors. JCI Insight. 2019; 4(22). doi: 10.1172/jci.insight.131610. PubMed PMID: 31723052; PMCID: PMC6948855.
87. Paulk N K, Pekrun K, Zhu E, Nygaard S, Li B, Xu J, Chu K, Leborgne C, Dane A P, Haft A, Zhang Y, Zhang F, Morton C, Valentine M B, Davidoff A M, Nathwani A C, Mingozzi F, Grompe M, Alexander I E, Lisowski L, Kay M A. Bioengineered AAV Capsids with Combined High Human Liver Transduction In Vivo and Unique Humoral Seroreactivity. Mol Ther. 2018; 26(1):289-303. Epub 2017/10/23. doi: 10.1016/j.ymthe.2017.09.021. PubMed PMID: 29055620; PMCID: PMC5763027.
88. Ogden P J, Kelsic E D, Sinai S, Church G M. Comprehensive AAV capsid fitness landscape reveals a viral gene and enables machine-guided design. Science. 2019; 366(6469):1139-43. doi: 10.1126/science.aaw2900. PubMed PMID: 31780559.
89. Li C, Diprimio N, Bowles D E, Hirsch M L, Monahan P E, Asokan A, Rabinowitz J, Agbandje-McKenna M, Samulski R J. Single amino acid modification of adeno-associated virus capsid changes transduction and humoral immune profiles. J Virol. 2012; 86(15):7752-9. Epub 2012/05/18. doi: JVI.00675-12 [pii]10.1128/JVI.00675-12. PubMedPMID: 22593151; PMCID: 3421647.
90. Huttner N A, Girod A, Perabo L, Edbauer D, Kleinschmidt J A, Buning H, Hallek M. Genetic modifications of the adeno-associated virus type 2 capsid reduce the affinity and the neutralizing effects of human serum antibodies. Gene Ther. 2003; 10(26):2139-47. doi: 10.1038/sj.gt.3302123. PubMed PMID: 14625569.
91. Steines B, Dickey D D, Bergen J, Excoffon K J, Weinstein J R, Li X, Yan Z, Abou Alaiwa M H, Shah V S, Bouzek D C, Powers L S, Gansemer N D, Ostedgaard L S, Engelhardt J F, Stoltz D A, Welsh M J, Sinn P L, Schaffer D V, Zabner J. CFTR gene transfer with AAV improves early cystic fibrosis pig phenotypes. JCI Insight. 2016; 1(14):e88728. Epub 2016/10/05. doi: 10.1172/jci.insight.88728. PubMed PMID: 27699238; PMCID: PMC5033908.
92. Marsic D, Govindasamy L, Currlin S, Markusic D M, Tseng Y S, Herzog R W, Agbandje-McKenna M, Zolotukhin S. Vector design Tour de Force: integrating combinatorial and rational approaches to derive novel adeno-associated virus variants. Mol Ther. 2014; 22(11):1900-9. Epub 2014/07/23. doi: 10.1038/mt.2014.139 mt2014139 [pii]. PubMed PMID: 25048217.
93. Bates E, Miller S, Alexander M, Mazur M, Fortenberry J A, Bebok Z, Sorscher E J, Rowe S M. Bioelectric effects of quinine on polarized airway epithelial cells. Journal of cystic fibrosis: official journal of the European Cystic Fibrosis Society. 2007; 6(5):351-9. Epub 2007/03/03. doi: 10.1016/j.jcf.2007.01.001. PubMed PMID: 17329172; PMCID: 2077327.
94. Nathwani A C, Davidoff A M, Hanawa H, Hu Y, Hoffer F A, Nikanorov A, Slaughter C, Ng C Y, Zhou J, Lozier J N, Mandrell T D, Vanin E F, Nienhuis A W. Sustained high-level expression of human factor IX (hFIX) after liver-targeted delivery of recombinant adeno-associated virus encoding the hFIX gene in rhesus macaques. Blood. 2002; 100(5):1662-9. doi: 10.1182/blood-2002-02-0589. PubMed PMID: 12176886.
95. Duncan G A, Jung J, Joseph A, Thaxton A L, West N E, Boyle M P, Hanes J, Suk J S. Microstructural alterations of sputum in cystic fibrosis lung disease. JCI Insight. 2016; 1(18):e88198. doi: 10.1172/jci.insight.88198. PubMed PMID: 27812540; PMCID: PMC5085601 developed by Kala Pharmaceuticals. J. Hanes is a cofounder of Kala Pharmaceuticals. He owns company stock, which is subject to certain restrictions under Johns Hopkins University policy. The terms of this arrangement are being managed by Johns Hopkins University in accordance with its conflict-of-interest policies.
96. Suk J S, Kim A J, Trehan K, Schneider C S, Cebotaru L, Woodward O M, Boylan N J, Boyle M P, Lai S K, Guggino W B, Hanes J. Lung gene therapy with highly compacted DNA nanoparticles that overcome the mucus barrier. J Control Release. 2014; 178C:8-17. Epub 2014/01/21. doi: 10.1016/j.jconrel.2014.01.007. PubMed PMID: 24440664.
97. Akil O, Dyka F, Calvet C, Emptoz A, Lahlou G, Nouaille S, Boutet de Monvel J, Hardelin J P, Hauswirth W W, Avan P, Petit C, Safieddine S, Lustig L R. Dual AAV-mediated gene therapy restores hearing in a DFNB9 mouse model. Proc Natl Acad Sci USA. 2019. Epub 2019/02/21. doi: 10.1073/pnas.1817537116. PubMed PMID: 30782832; PMCID: PMC6410774.
98. Al-Moyed H, Cepeda A P, Jung S, Moser T, Kugler S, Reisinger E. A dual-AAV approach restores fast exocytosis and partially rescues auditory function in deaf otoferlin knock-out mice. EMBO Mol Med. 2019; 11(1). Epub 2018/12/05. doi: 10.15252/emmm.201809396. PubMed PMID: 30509897; PMCID: PMC6328916.
99. Carvalho L S, Turunen H T, Wassmer S J, Luna-Velez M V, Xiao R, Bennett J, Vandenberghe L H. Evaluating Efficiencies of Dual AAV Approaches for Retinal Targeting. Front Neurosci. 2017; 11:503. Epub 2017/09/26. doi: 10.3389/fnins.2017.00503. PubMed PMID: 28943836; PMCID: PMC5596095.
100. Kim Y C, Hsueh H T, Kim N, Rodriguez J, Leo K T, Rao D, West N E, Hanes J, Suk J S. Strategy to enhance dendritic cell-mediated DNA vaccination in the lung. Adv Ther (Weinh). 2021; 4(2). Epub 2021/03/13. doi: 10.1002/adtp.202000228. PubMed PMID: 33709020; PMCID: PMC7941873.
101. Krotova K, Aslanidi G. Modifiers of Adeno-Associated Virus-Mediated Gene Expression in Implication for Serotype-Universal Neutralizing Antibody Assay. Hum Gene Ther. 2020; 31(19-20):1124-31. doi: 10.1089/hum.2020.074. PubMed PMID: 32495655; PMCID: PMC7588322.
102. Krotova K, Day A, Aslanidi G. An Engineered AAV6-Based Vaccine Induces High Cytolytic Anti-Tumor Activity by Directly Targeting DCs and Improves Ag Presentation. Mol Ther Oncolytics. 2019; 15:166-77. doi: 10.1016/j.omto.2019.10.001. PubMed PMID: 31720373; PMCID: PMC6838889.

### Other Embodiments

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

1. A method of delivery of nucleic acid, through a mucus gel layer comprising administering a composition comprising a virus vector associated with an extracellular vesicle, wherein the virus vector comprises the nucleic acid.

2. The method of claim 1, wherein the virus vector is associated externally with the extracellular vesicle and/or is contained within the extracellular vesicle.

3. The method of claim 1, wherein the virus vector comprises: adenovirus vectors, adeno-associated virus (AAV) vectors, herpes simplex virus vectors, influenza virus vectors, lentivirus vector, retrovirus vectors, alphavirus vectors, flavivirus vectors, rhabdovirus vectors, measles virus vectors, Newcastle disease virus vectors, poxvirus vectors, vaccinia virus vectors, modified Ankara virus vectors, vesicular stomatitis virus vectors, picornavirus vectors, chimeric virus vectors, synthetic virus vectors, respiratory syncytial virus vectors, parainfluenza virus vectors, foamy virus vectors, recombinant viral vectors, mosaic virus vectors, or pseudotyped virus vectors.

4. The method of claim 3, wherein the virus vector comprises one or more mutations.

5. The method of claim 3, wherein the virus vector is an AAV vector.

6. The method of claim 5, wherein the AAV vector comprises one or more AAV capsid protein(s) comprising modified amino acid sequences.

7-11. (canceled)

12. The method of claim 1, wherein the extracellular vesicle is derived from eukaryotic cells.

13. The method of claim 1, wherein the composition is formulated for oral, intratracheal/oropharyngeal cervicovaginal, ocular, rectal or nasal delivery.

14-16. (canceled)

17. A composition comprising a virus vector associated with an extracellular vesicle, wherein the virus vector comprises a transgene.

18. The composition of claim 17, wherein the virus vector is associated externally with the extracellular vesicle or is contained within the extracellular vesicle.

19. The composition of claim 17, wherein the virus vector comprises: adenovirus vectors, adeno-associated virus (AAV) vectors, herpes simplex virus vectors, influenza virus vectors, lentivirus vector, retrovirus vectors, alphavirus vectors, flavivirus vectors, rhabdovirus vectors, measles virus vectors, Newcastle disease virus vectors, poxvirus vectors, vaccinia virus vectors, modified Ankara virus vectors, vesicular stomatitis virus vectors, picornavirus vectors, chimeric virus vectors, synthetic virus vectors, respiratory syncytial virus vectors, parainfluenza virus vectors, foamy virus vectors, recombinant viral vectors, mosaic virus vectors, or pseudotyped virus vectors.

20. The composition of claim 19, wherein the virus vector is an AAV vector.

21. The composition of claim 20, wherein the AAV vector comprises one or more AAV capsid protein(s) comprising modified amino acid sequences.

22-28. (canceled)

29. The composition of claim 17, wherein the extracellular vesicle is a modified extracellular vesicle derived from a cell transduced with an exogenous nucleic acid sequence.

30-32. (canceled)

33. A device for delivery of the composition of claim 17 to an oral cavity, nose, pharynx or to a subject in need thereof.

34. A method of treating a lung disease or disorder or an oropharyngeal disease comprising administering the composition of claim 17 to a subject in need thereof.

35. The method of claim 34, wherein a lung disease or disorder comprises cystic fibrosis, chronic obstructive pulmonary disease (COPD), lung inflammation, asthma, lung cancer, bronchitis, infections, or allergies.

36. A method of modulating an immune response for treating an immune related disorder in a subject in need thereof, comprising administering a composition comprising an extracellular vesicle and an adeno-associated virus (AAV) vector, the AAV vector comprising a nucleic acid sequence encoding an immunomodulatory molecule.

37. The method of claim 36, wherein the AAV comprises one or more amino acid mutations in the AAV capsid proteins.

38-49. (canceled)

50. An adeno-associated virus (AAV) vector comprising one or more amino acid mutations in the AAV capsid proteins, or
a composition comprising a modified extracellular vesicle and a virus vector comprising a transgene, or
a nucleic acid-based vaccine comprising an extracellular vesicle and a virus vector wherein the virus vector comprises at least one transgene.

51-69. (canceled)
